SEQUENCE LISTING (138 sequences)

SEQ ID NO: 1 - protein, 5 aa, synthetic construct
SYWIH

SEQ ID NO: 2 - protein, 5 aa, synthetic construct
DYYMS

SEQ ID NO: 3 - protein, 10 aa, synthetic construct
GFTFSDYYMY

SEQ ID NO: 4 - protein, 10 aa, synthetic construct
GYTFTDYSLH

SEQ ID NO: 5 - protein, 10 aa, synthetic construct
GYTFTNYGMN

SEQ ID NO: 6 - protein, 8 aa, synthetic construct
GFTDYYMS

SEQ ID NO: 7 - protein, 10 aa, synthetic construct
GYTFTDYGMN

SEQ ID NO: 8 - protein, 5 aa, synthetic construct
AYTMT

SEQ ID NO: 9 - protein, 10 aa, synthetic construct
EITFIDYYMS

SEQ ID NO: 10 - protein, 10 aa, synthetic construct
GYTFTDFAMH

SEQ ID NO: 11 - protein, 10 aa, synthetic construct
GYTFTDFAIH

SEQ ID NO: 12 - protein, 5 aa, synthetic construct
DYFMS

SEQ ID NO: 13 - protein, 5 aa, synthetic construct
TNAMN

SEQ ID NO: 14 - protein, 5 aa, synthetic construct
SYWIN

SEQ ID NO: 15 - protein, 17 aa, synthetic construct
RIYPGTGSTYYNEKFKG

SEQ ID NO: 16 - protein, 19 aa, synthetic construct
YIRNKVNGHTTDYIASVKG

SEQ ID NO: 17 - protein, 17 aa, synthetic construct
TISDAGTYTSYLDSVKG

SEQ ID NO: 18 - protein, 17 aa, synthetic construct
WINTETGEPTYADDFKG

SEQ ID NO: 19 - protein, 17 aa, synthetic construct
WINTYTGKPTYADDIKE

SEQ ID NO: 20 - protein, 19 aa, synthetic construct
FISNKANGFTTDYSASVKG

SEQ ID NO: 21 - protein, 17 aa, synthetic construct
WINTYTGEPTYPDDFKG

SEQ ID NO: 22 - protein, 17 aa, synthetic construct
LIAPYHGGTVYNQKFKG

SEQ ID NO: 23 - protein, 19 aa, synthetic construct
LIRNKANGYTTEYSESVKG

SEQ ID NO: 24 - protein, 17 aa, synthetic construct
IITTYGGDTNYNQKFRG

SEQ ID NO: 25 - protein, 19 aa, synthetic construct
LIRNKANGYIIEYSESVKG

SEQ ID NO: 26 - protein, 19 aa, synthetic construct
LIRNKVNGYTIEYSASVKG

SEQ ID NO: 27 - protein, 19 aa, synthetic construct
RIRSKSNNYATYYADSVKD

SEQ ID NO: 28 - protein, 13 aa, synthetic construct
NIYPYTSSSNYNE

SEQ ID NO: 29 - protein, 12 aa, synthetic construct
SGFPLTTVPLDY

SEQ ID NO: 30 - protein, 10 aa, synthetic construct
DPGINWYFDV

SEQ ID NO: 31 - protein, 12 aa, synthetic construct
NYYGSSPYGMDY

SEQ ID NO: 32 - protein, 11 aa, synthetic construct
PVSLLFYYFTV

SEQ ID NO: 33 - protein, 10 aa, synthetic construct
RDYRGWYFDV

SEQ ID NO: 34 - protein, 10 aa, synthetic construct
DIGIRYYFAY

SEQ ID NO: 35 - protein, 9 aa, synthetic construct
GDLLVRLDN

SEQ ID NO: 36 - protein, 12 aa, synthetic construct
GASSGFVTWFAY

SEQ ID NO: 37 - protein, 12 aa, synthetic construct
NRAYRFFYAMDY

SEQ ID NO: 38 - protein, 10 aa, synthetic construct
DIGIRWNFDV

SEQ ID NO: 39 - protein, 10 aa, synthetic construct
DIGIRWYFDV

SEQ ID NO: 40 - protein, 7 aa, synthetic construct
FLSLMDY

SEQ ID NO: 41 - protein, 15 aa, synthetic construct
KGNIYYSYESYPLDF

SEQ ID NO: 42 - protein, 11 aa, synthetic construct
RASKSISKYLA

SEQ ID NO: 43 - protein, 10 aa, synthetic construct
RASSRVTYMH

SEQ ID NO: 44 - protein, 10 aa, synthetic construct
SASSSVIYMH

SEQ ID NO: 45 - protein, 11 aa, synthetic construct
RASQDISHFLN

SEQ ID NO: 46 - protein, 15 aa, synthetic construct
RASESVDIYAVSFMS

SEQ ID NO: 47 - protein, 10 aa, synthetic construct
RASSSVTYMH

SEQ ID NO: 48 - protein, 11 aa, synthetic construct
RASQSVNNDVV

SEQ ID NO: 49 - protein, 11 aa, synthetic construct
KASQDINKYIS

SEQ ID NO: 50 - protein, 11 aa, synthetic construct
GASENIYGALN

SEQ ID NO: 51 - protein, 10 aa, synthetic construct
RASSSVSYMH

SEQ ID NO: 52 - protein, 10 aa, synthetic construct
SATSRVTYMH

SEQ ID NO: 53 - protein, 10 aa, synthetic construct
SASSSISYMH

SEQ ID NO: 54 - protein, 11 aa, synthetic construct
KASEDIYKQIG

SEQ ID NO: 55 - protein, 7 aa, synthetic construct
SGSTLQS

SEQ ID NO: 56 - protein, 7 aa, synthetic construct
ATSNLAS

SEQ ID NO: 57 - protein, 7 aa, synthetic construct
DTSKLAS

SEQ ID NO: 58 - protein, 7 aa, synthetic construct
YTSRLLS

SEQ ID NO: 59 - protein, 7 aa, synthetic construct
LASKQGS

SEQ ID NO: 60 - protein, 7 aa, synthetic construct
ATSSLAS

SEQ ID NO: 61 - protein, 7 aa, synthetic construct
CASNRYT

SEQ ID NO: 62 - protein, 7 aa, synthetic construct
YTSTLQS

SEQ ID NO: 63 - protein, 7 aa, synthetic construct
GATNLAD

SEQ ID NO: 64 - protein, 7 aa, synthetic construct
ATSTLAS

SEQ ID NO: 65 - protein, 7 aa, synthetic construct
GATSLET

SEQ ID NO: 66 - protein, 9 aa, synthetic construct
QQHNEYPFT

SEQ ID NO: 67 - protein, 9 aa, synthetic construct
QQWSYNPPT

SEQ ID NO: 68 - protein, 9 aa, synthetic construct
QQWSSHPLT

SEQ ID NO: 69 - protein, 9 aa, synthetic construct
QQGKTLPWT

SEQ ID NO: 70 - protein, 9 aa, synthetic construct
QQSKEVPWT

SEQ ID NO: 71 - protein, 9 aa, synthetic construct
QQDYRSPWT

SEQ ID NO: 72 - protein, 8 aa, synthetic construct
LQYDYLRT

SEQ ID NO: 73 - protein, 9 aa, synthetic construct
QNVLSTPYT

SEQ ID NO: 74 - protein, 9 aa, synthetic construct
QRWGYNPPT

SEQ ID NO: 75 - protein, 9 aa, synthetic construct
QNVLFAPYT

SEQ ID NO: 76 - protein, 9 aa, synthetic construct
QSVLFAPYT

SEQ ID NO: 77 - protein, 9 aa, synthetic construct
QQWSGNPPT

SEQ ID NO: 78 - protein, 9 aa, synthetic construct
HQRSSYPWT

SEQ ID NO: 79 - protein, 9 aa, synthetic construct
QQYWSPPFT

SEQ ID NO: 80 - protein, 121 aa, synthetic construct
QVQLKQSGAELVRPGASVKLSCKTSGYIFTSYWIHWVKQRSGQGLEWIARIYPGTGSTYYNEKFKGKATLTADKSSSTAYMQLSSLKSEDSAVYFCARSGFPLTTVPLDYWGQGTTLTVSS

SEQ ID NO: 81 - protein, 121 aa, synthetic construct
EVKLVESGGGLVQPGGSLRLSCATSEFTLIDYYMSWVRQSPGKALEWLGYIRNKVNGHTTDYIASVKGRFAISRDNSQSILYLQMNTLRVEDSATYYCVRDPGINWYFDVWGAGTTVTVSS

SEQ ID NO: 82 - protein, 121 aa, synthetic construct
EVQMVESGGGLVKPGGSLKLSCAASGFTFSDYYMYWVRQSPEKRLEWVATISDAGTYTSYLDSVKGRFIISRDNAKNNLYLQMSSLKSEDTAIYYCARNYYGSSPYGMDYWGQGTSVTVSS

SEQ ID NO: 83 - protein, 120 aa, synthetic construct
QIQLVQSGPELKKPGETVKISCKASGYTFTDYSLHWVKQVPVKGLKWMGWINTETGEPTYADDFKGRFAFSLDTSASSAYLQIHNLKNEDTATYFCASPVSLLFYYFTVWGAGTTVTVSA

SEQ ID NO: 84 - protein, 119 aa, synthetic construct
QIQLVQSGPELMKPGETVKISCKASGYTFTNYGMNWVKQAPGKGLKWLGWINTYTGKPTYADDIKERFVFSLETSASTAYLQINNLKNEDTATYFCARRDYRGWYFDVWGAGTPVTVSS

SEQ ID NO: 85 - protein, 119 aa, synthetic construct
EVRLVESGGDLVQPGGSLRLSCTTSGFTDYYMSWVRQPPGKALEWLGFISNKANGFTTDYSASVKGRFTISRDNSRSILYLQMNTLRPEDSATYYCAGDIGIRYYFAYWGQGTTLTVSS

SEQ ID NO: 86 - protein, 118 aa, synthetic construct
QIQLVQSGPELKKPGETVKISCKASGYTFTDYGMNWVKQAPGKGLKWMGWINTYTGEPTYPDDFKGRFAFSLETSATTAYLQINNLKNEDTATYFCSRGDLLVRLDNWGQGTTLTVSS

SEQ ID NO: 87 - protein, 121 aa, synthetic construct
EVQLRQSGPELVRPGASMKISCKASGYSFTAYTMTWVKQSHGKILEWIGLIAPYHGGTVYNQKFKGKATLTVDKSSSTAYMELLSLTSEDSAIYYCAGGASSGFVTWFAYWGQGTLVTVSA

SEQ ID NO: 88 - protein, 121 aa, synthetic construct
EVKLVESGGGLVQPGGSLRLSCATSEITFIDYYMSWVRQPPGKALEWLGLIRNKANGYTTEYSESVKGRFTISRDKSQSILYLQMNTLRVEDSATYYCARDPGINWYFDVWGAGTTVTVSS

SEQ ID NO: 89 - protein, 121 aa, synthetic construct
QVQLQQSGPELVRPGVSVKISCKGSGYTFTDFAMHWVRQSHSKSLEWVGIITTYGGDTNYNQKFRGKATMTVDRSSSTVFLELARLTSEDSAIYYCARNRAYRFFYAMDYWGQGTSVTVSS

SEQ ID NO: 90 - protein, 121 aa, synthetic construct
QVQLQQSGPELVRPGVSVKISCKGSGYTFTDFAIHWVRQSHSRSLEWVGIITTYGGDTNYNQKFRGKATMTVDKSSSTVFLELARLTSEDSAIYYCARNRAYRFFYAMDYWGQGTSVTVSS

SEQ ID NO: 91 - protein, 121 aa, synthetic construct
EVKLVESGGGLVQPGGSLSLSCAVSGFIFTDYFMSWVRQPPGKTLEWLALIRNKANGYIIEYSESVKGRFTISRGNSQSIFYLQMNALRTEDSATYYCARDIGIRWNFDVWGAGTTVTVSS

SEQ ID NO: 92 - protein, 121 aa, synthetic construct
EVKLVESGGGLVQPGGSLSLSCTASGFTLTDYYMSWVRQPPGRALEWLALIRNKVNGYTIEYSASVKGRFTISRDNSQSILYLQMNALRAEDSATYYCARDIGIRWYFDVWGAGTTVTVSS

SEQ ID NO: 93 - protein, 118 aa, synthetic construct
EVQLVETGGGLVQPKGSLKLSCAASGFSFNTNAMNWVRQAPGKGLEWVARIRSKSNNYATYYADSVKDRFTISRDDSQSMLYLQMNNLKTEDTAMYYCVSFLSLMDYWGQGTSVTVSS

SEQ ID NO: 94 - protein, 124 aa, synthetic construct
QVQLQQPGAELVKPGASVTLSCKASGYTFTSYWINWVKQRPGQGLEWIGNIYPYTSSSNYNEKFKGKATLTVDTSSNTAYMQLSGLTSDDSAVYFCARKGNIYYSYESYPLDFWGQGTSVTVSS

SEQ ID NO: 95 - protein, 107 aa, synthetic construct
DVQITQSPSYLAASPGETITINCRASKSISKYLAWYQEKPGKTNKLLIYSGSTLQSGIPSRFSGSGSGTDFTLTISSLEPEDFAMYYCQQHNEYPFTFGSGTKLEIK

SEQ ID NO: 96 - protein, 106 aa, synthetic construct
QIVLSQSPAILSASPGEKVTMTCRASSRVTYMHWYQQKPGSSPKPWISATSNLASGVPARFSGSGSGTSYSLTISRVEAEDAATYYCQQWSYNPPTFGAGTKLELN

SEQ ID NO: 97 - protein, 106 aa, synthetic construct
QIVLTQSPAIMSASPGEKVTMTCSASSSVIYMHWYQQKSGTSPKRWIYDTSKLASGVPARFSGSGSGTSYSLTISSMEAEDAATYYCQQWSSHPLTFGAGTKLELK

SEQ ID NO: 98 - protein, 107 aa, synthetic construct
DIQMTQTTSSLSASLGDRVTISCRASQDISHFLNWYQQKPDGTVELLIYYTSRLLSGVPSRFSGSGSGTDYSLTIANLEQEDIATYFCQQGKTLPWTFGGGTKVEIK

SEQ ID NO: 99 - protein, 111 aa, synthetic construct
DIVLTQSPASLAVSLGQRATISCRASESVDIYAVSFMSWFQQKPGQPPKLLIYLASKQGSGVPARFSGSGSGTEFSLNIHPMEEDDAAMYFCQQSKEVPWTFGGGTKLEIK

SEQ ID NO: 100 - protein, 106 aa, synthetic construct
QIVLSQSPAILSASPGERVTMTCRASSSVTYMHWYQQKPGSSPKSWIYATSSLASGVPARFSGSGSGTSYSLTISRVEAEDAATYYCQQWSYNPPTFGAGTKLELK

SEQ ID NO: 101 - protein, 107 aa, synthetic construct
AIVMTQTPKFLLISAGDRINITCRASQSVNNDVVWYQQKPGQSPKLLIYCASNRYTGVPDRFTGSGYGTDLTFNISTVQAEDPAVYFCQQDYRSPWTFGGGTKLEIK

SEQ ID NO: 102 - protein, 106 aa, synthetic construct
DIQMTQSPSSLSASLGGKVTITCKASQDINKYISWYQHKPGKGPRLLIHYTSTLQSGIPSRFSGSGSGRDYSFSISNLEPEDIATYYCLQYDYLRTFGGGTKLEIK

SEQ ID NO: 103 - protein, 107 aa, synthetic construct
DIQMTQSPASLSASVGETVTITCGASENIYGALNWYQRKQGKSPQVLIYGATNLADGTSSRFSGSGSGRQYSLKINSLHPDDVATYYCQNVLSTPYTFGGGTKLEIK

SEQ ID NO: 104 - protein, 106 aa, synthetic construct
QNILSQSPTILSASPGEKVTMTCRASSSVSYMHWYQQKPGSSPKPWIYATSNLASGVPARFSGSGSGTSYSLTISRVEAEDVAIYFCQRWGYNPPTFGAGTKLELK

SEQ ID NO: 105 - protein, 107 aa, synthetic construct
DIQMTQSPASLSASVGETVTITCGASENIYGALNWYQRKQGKSPHLLIYGATNLADGMSSRFSGSGSAGQYSLNISSLHPDDVATYYCQNVLFAPYTFGGGTRLEIK

SEQ ID NO: 106 - protein, 107 aa, synthetic construct
DIQMTQSPASLSASVGETVTITCGASENIYGALNWYQRKQGKSPQLLIYGATNLADGMSSRFSGSGSAGQYSLNISSLHPDDVATYYCQSVLFAPYTFGGGTRLEIK

SEQ ID NO: 107 - protein, 106 aa, synthetic construct
QIVLTQSPAIMSASPGEKVTMTCSATSRVTYMHWYQQKSGTSPKRWIYATSTLASGVPARFSGSGSGTSYSLTISSMEAEDAATYYCQQWSYNPPTFGSGTKLEIK

SEQ ID NO: 108 - protein, 106 aa, synthetic construct
QIVLSQSPAILSASPGEKVTMTCRASSSVSYMHWYQQKPGSSPKPWIYATSNLASGVPARFSGSGSGTSYSLTISRMEAEDAATYYCQQWSGNPPTFGGGTKLEIK

SEQ ID NO: 109 - protein, 106 aa, synthetic construct
QIVLTQSPAIMSASPGEKVTMTCSASSSISYMHWYQQKPGTSPKRWIYDTSKLASGVPARFSGSGSGTSYSLTISSMEAEDAATYYCHQRSSYPWTFGGGTKLEIK

SEQ ID NO: 110 - protein, 107 aa, synthetic construct
DIQMTQSSSSFSVSLGDRVTITCKASEDIYKQIGWYQQKPGNAPRLLIAGATSLETGVPSRFSGSGSGKDYMLNITSLQTEDVGSYYCQQYWSPPFTFGSGTKLEIK

SEQ ID NO: 111 - protein, 121 aa, synthetic construct
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSS

SEQ ID NO: 112 - protein, 107 aa, synthetic construct
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIK

SEQ ID NO: 113 - protein, 451 aa, synthetic construct
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 214 aa, synthetic construct
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 330 aa, synthetic construct [sequence omitted]

SEQ ID NO: 116 - protein, 107 aa, synthetic construct [sequence omitted]

SEQ ID NO: 117 - protein, 330 aa, synthetic construct
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 118 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 119 - protein, 451 aa, synthetic construct
QVQLVQSGAEVKKPGASVKVSCKASGYTFTDYAMHWVRQAPGQRLEWMGLISTYSGDTKYNQNFQGRVTMTVDKSASTAYMELSSLRSEDTAVYYCARGDYSGSRYWFAYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 120 - protein, 214 aa, synthetic construct
DIQMTQSPSSLSASVGDRVTITCQASENIYGALNWYQRKPGKSPKLLIYGASNLATGMPSRFSGSGSGTDYTFTISSLQPEDIATYYCQQVLSSPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 121 - protein, 19 aa, synthetic construct
YIRNKVQGHTTDYIASVKG

SEQ ID NO: 122 - protein, 121 aa, synthetic construct
EVQLVESGGGLVQPGGSLRLSCAASEFTLIDYYMSWVRQAPGKGLEWVGYIRNKVNGHTTDYIASVKGRFTISRDDSKNSLYLQMNSLKTEDTAVYYCARDPGINWYFDVWGQGTLVTVSS

SEQ ID NO: 123 - protein, 121 aa, synthetic construct
EVQLVESGGGLVQPGGSLRLSCATSEFTLIDYYMSWVRQAPGKALEWVGYIRNKVNGHTTDYIASVKGRFTISRDDSKNSLYLQMNSLKTEDTAVYYCVRDPGINWYFDVWGQGTLVTVSS

SEQ ID NO: 124 - protein, 121 aa, synthetic construct
EVKLVESGGGLVQPGGSLRLSCATSEFTLIDYYMSWVRQSPGKALEWLGYIRNKVNGHTTDYIASVKGRFTISRDDSKNSLYLQMNSLKTEDTATYYCVRDPGINWYFDVWGQGTLVTVSS

SEQ ID NO: 125 - protein, 121 aa, synthetic construct
EVKLVESGGGLVQPGGSLRLSCATSEFTLIDYYMSWVRQSPGKALEWLGYIRNKVQGHTTDYIASVKGRFTISRDDSKNSLYLQMNSLKTEDTATYYCVRDPGINWYFDVWGQGTLVTVSS

SEQ ID NO: 126 - protein, 106 aa, synthetic construct
EIVLTQSPATLSLSPGERATLSCRASSRVTYMHWYQQKPGQAPRLLIYATSNLASGIPARFSGSGSGTDFTLTISSLEPEDFAVYYCQQWSYNPPTFGGGTKLELK

SEQ ID NO: 127 - protein, 106 aa, synthetic construct
EIVLTQSPATLSLSPGERATLSCRASSRVTYMHWYQQKPGQSPRPLISATSNLASGIPARFSGSGSGTDYTLTISSLEPEDFAVYYCQQWSYNPPTFGGGTKLELK

SEQ ID NO: 128 - protein, 106 aa, synthetic construct
QIVLTQSPATLSLSPGERATLSCRASSRVTYMHWYQQKPGSSPKPLISATSNLASGIPARFSGSGSGTDYTLTISSLEPEDFATYYCQQWSYNPPTFGGGTKLELK

SEQ ID NO: 129 - protein, 451 aa, synthetic construct
EVQLVESGGGLVQPGGSLRLSCAASEFTLIDYYMSWVRQAPGKGLEWVGYIRNKVNGHTTDYIASVKGRFTISRDDSKNSLYLQMNSLKTEDTAVYYCARDPGINWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 451 aa, synthetic construct
EVQLVESGGGLVQPGGSLRLSCATSEFTLIDYYMSWVRQAPGKALEWVGYIRNKVNGHTTDYIASVKGRFTISRDDSKNSLYLQMNSLKTEDTAVYYCVRDPGINWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 451 aa, synthetic construct
EVKLVESGGGLVQPGGSLRLSCATSEFTLIDYYMSWVRQSPGKALEWLGYIRNKVNGHTTDYIASVKGRFTISRDDSKNSLYLQMNSLKTEDTATYYCVRDPGINWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 451 aa, synthetic construct
EVKLVESGGGLVQPGGSLRLSCATSEFTLIDYYMSWVRQSPGKALEWLGYIRNKVQGHTTDYIASVKGRFTISRDDSKNSLYLQMNSLKTEDTATYYCVRDPGINWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 213 aa, synthetic construct
EIVLTQSPATLSLSPGERATLSCRASSRVTYMHWYQQKPGQAPRLLIYATSNLASGIPARFSGSGSGTDFTLTISSLEPEDFAVYYCQQWSYNPPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 213 aa, synthetic construct
EIVLTQSPATLSLSPGERATLSCRASSRVTYMHWYQQKPGQSPRPLISATSNLASGIPARFSGSGSGTDYTLTISSLEPEDFAVYYCQQWSYNPPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 213 aa, synthetic construct
QIVLTQSPATLSLSPGERATLSCRASSRVTYMHWYQQKPGSSPKPLISATSNLASGIPARFSGSGSGTDYTLTISSLEPEDFATYYCQQWSYNPPTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 451 aa, synthetic construct
EVKLVESGGGLVQPGGSLRLSCATSEFTLIDYYMSWVRQSPGKALEWLGYIRNKVNGHTTDYIASVKGRFAISRDNSQSILYLQMNTLRVEDSATYYCVRDPGINWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 213 aa, synthetic construct
QIVLSQSPAILSASPGEKVTMTCRASSRVTYMHWYQQKPGSSPKPWISATSNLASGVPARFSGSGSGTSYSLTISRVEAEDAATYYCQQWSYNPPTFGAGTKLELNRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 176 aa, synthetic construct
PKPSISSNNSNPVEDKDAVAFTCEPEVQNTTYLWWVNGQSLPVSPRLQLSNGNMTLTLLSVKRNDAGSYECEIQNPASANRSDPVTLNVLYGPDVPTISPSKANYRPGENLNLSCHAASNPPAQYSWFINGTFQQSTQELFIPNITVNNSGSYMCQAHNSATGLNRTTVTHHHHHH

(19) United States
(12) Patent Application Publication
(10) Pub. No.: US 2025/0250335 A1
(43) Pub. Date: Aug. 7, 2025

(54) ANTIBODY AGAINST CEACAM5 AND CEACAM6, AND USE THEREOF

(71) Applicant: SHENGHE (CHINA) BIOPHARMACEUTICAL CO., LTD., Nanjing, Jiangsu (CN)

(72) Inventors: Chong ZHOU, Nanjing (CN); Xiaoling JIANG, Nanjing (CN); Liusong YIN, Nanjing (CN); Chunling QIN, Nanjing (CN); Jinhua ZHOU, Nanjing (CN); Chongbing WU, Nanjing (CN)

(73) Assignee: SHENGHE (CHINA) BIOPHARMACEUTICAL CO., LTD., Nanjing, Jiangsu (CN)

(21) Appl. No.: 18/856,162

(22) PCT Filed: Apr. 11, 2023
(86) PCT No.: PCT/CN2023/087487

§ 371 Date: Oct. 11, 2024

(30) Foreign Application Priority Data
Apr. 12, 2022 (WO) PCT/CN2022/086315

(51) Int. Cl. C07K 16/28; C07K 16/30

(52) U.S. Cl. CPC C07K 16/2803; C07K 16/3007; C07K 2317/24; C07K 2317/31; C07K 2317/33; C07K 2317/732; C07K 2317/92

(57) ABSTRACT

An antibody against CEACAM5 and CEACAM6 or antigen-binding fragment thereof, and the use thereof. The antibody is an antibody against CEACAM5 and CEACAM6, which antibody has a good performance, and can specifically bind to both targets CEACAM5 and CEACAM6 with a high affinity, thereby expanding antibody indications. The antibody against CEACAM5 and CEACAM6 has a binding specificity, hardly binds to normal cells, and only binds to overexpressed tumor cells, thereby reducing adverse effects caused by non-specific binding. The antibody also has a good endocytosis activity, and can be used for coupling a cytotoxic drug to form an antibody-drug conjugate for anti-tumor therapy.

### TECHNICAL FIELD

The present invention belongs to the field of biomedicine and specifically relates to an antibody against CEACAM5 and CEACAM6 and use thereof.

### BACKGROUND

CEACAM belongs to the immunoglobulin superfamily of adhesion molecules, whose structural domains are highly glycosylated and usually include 1-2 immunoglobulin variable region-like structural domains (Ndomain) and 0-6 immunoglobulin constant region-like structural domains. CEACAM relates to a variety of cellular functions, is based on intercellular adhesion function, regulates cell growth and differentiation by signaling, and plays an important role in insulin homeostasis, angiogenesis, and immunomodulation. In humans, the CEACAM subgroup consists of seven members: CEACAM1, CEACAM3, CEACAM4, CEACAM5, CEACAM6, CEACAM7, and CEACAM8. Members of the CEACAM gene family are involved in a wide variety of pathophysiologic roles, including as receptors for microbial pathogens, and they play important roles in carcinogenesis, cancer detection, progression, and metastasis.

CEACAM5 (abbreviated as CEA, also known as CD66e) is a glycoprotein with a molecular weight of approximately 180 kDa that encodes the CEA protein. CEACAM5 contains seven structural domains connected to the cell membrane via a glycosylphosphatidylinositol (GPI) anchor, and the seven structural domains include a single N-terminal Ig variable domain and six structural domains homologous to the Ig constant domain (A1-B1-A2-B2-A3-B3). CEACAM5 was first described as a gastrointestinal carcinoembryonic antigen in 1965, but to date, it has been shown to be highly expressed on the surface of colorectal, stomach, lung, breast, prostate, ovarian, uterine cervix, and bladder tumor cells and weakly expressed in a few normal epithelial tissues (Columnar epithelium and cup cells in the colon, mucus neck cells in the stomach, and squamous epithelial cells in the esophagus and uterine cervix). For example, in prostate and colorectal cancers, overexpression of CEACAM5 has been shown to serve as a tumor biomarker.

CEACAM6 (also known as CD66c or NCA-90) is a glycosylphosphatidylinositol (GPI)-linked cell-surface protein with one N- and 2 C2-like structural domains that mediates many cis- or trans-directed CEACAM interactions through its extracellular structural domains with a variety of membrane receptors (some of which have been identified). CEACAM6 is expressed on granulocytes and epithelial cells from a variety of organs and has a more extensive expression zone in proliferating cells of hyperplastic colon polyps and adenomas compared to normal mucosa, cancers, and relatively high serum levels of CEACAM6 have been found in patients with lung cancer, pancreatic cancer, breast cancer, colorectal cancer and hepatocellular carcinoma. Overexpression of CEACAM6 leads to morphologic alterations similar to epithelial-mesenchymal transition, resulting in increased invasiveness and chemoresistance. Previous studies have shown that tumor growth inhibition can be achieved by CEACAM6 silencing using CEACAM6-specific siRNA, and that inhibition of CEACAM6 function using antibody fragments can affect cell migration, cell invasion, and cell adhesion in vitro. These findings indicate that CEACAM6 is a good biomarker for various tumors.

In addition, CEACAM5/CEACAM6 have been found to be overexpressed in a variety of malignant tumors, such as in breast, pancreatic, ovarian, colon, lung and stomach tumors, and have been associated with tumor aggressiveness and metastasis.

The extracellular structural domains of CEACAM family members consist of repetitive immunoglobulin-like (Ig-like) structural domains, and the repetitive immunoglobulin-like (Ig-like) structural domains have been categorized into three types based on sequence homology: A, B and N. CEACAM5 contains seven such structural domains, namely N, A1, B1, A2, B2, A3 and B3. The CEACAM5 A1, A2, and A3 structural domains exhibit high sequence homology with the B1, B2, and B3 structural domains, with the A structural domain of human CEACAM5 showing 84% to 87% pairwise sequence similarity and the B structural domain showing 69% to 80% pairwise sequence similarity. In addition, other human CEACAM members (i.e., CEACAM1, CEACAM6, CEACAM7, and CEACAM8) presenting A and/or B structural domains in their structures demonstrate homology to human CEACAM5. Specifically, the A and B structural domains of the human CEACAM6 protein exhibit sequence homology with either of the A1 and A3 structural domains and the B1 to B3 structural domains of human CEACAM5, respectively, which is even higher than that observed in the A and B structural domains of human CEACAM5.

Due to the homology, some antibodies can show binding to duplicated epitopes of CEACAM5 present in different immunoglobulin structural domains, exhibiting cross-reactivity with other CEACAM members (e.g., CEACAM6). Due to CEACAM members have high expression in tumors, but different CEACAM members have expression differences in different tumors. Therefore, this cross-reactivity can increase the variety of tumors treated and expand the applicable therapeutic population. However, CEACAM members also have a certain expression level in normal tissues, and antibodies with cross-reactivity may pose a risk of binding to normal tissues, thereby reducing the therapeutic effect.

Therefore, it is necessary to develop anti-CEACAM5 and CEACAM6 antibodies, antigen-binding fragments, or fusion proteins that bind differentially to tumor tissues versus normal tissues (highly bound to tumor cells and weakly or not bound to normal cells) and that cross-react less with surrounding healthy tissues.

### SUMMARY

In response to the existing problems, the inventors have developed an antibody against CEACAM5 and CEACAM6, which antibody has a good performance, and can specifically bind to both targets CEACAM5 and CEACAM6 with a high affinity, thereby expanding antibody indications. At the same time, the antibody against CEACAM5 and CEACAM6 has a binding specificity, hardly binds to normal cells, and only binds to overexpressed tumor cells, thereby reducing adverse effects caused by non-specific binding.

The present invention provides an antibody against CEACAM5 and CEACAM6 or antigen-binding fragment thereof comprising a heavy chain variable region and a light chain variable region, wherein the heavy chain variable region comprises the heavy chain complementary determining regions HCDR1, HCDR2 and HCDR3, and the light chain variable region comprises the light chain complementary determining regions LCDR1, LCDR2 and LCDR3, wherein,
(a) HCDR1 of the heavy chain variable region is selected from any amino acid sequence of any of SEQ ID NO: 1-14, or a sequence having at least 80%, 85%, 90%, 91%, 92%, 93%, 94%, 95%, 96%, 97%, 98%, 99% or more identity to the amino acid sequence of any of SEQ ID NO: 1-14, or an amino acid sequence having one or more (preferably 2 or 3) conserved amino acid mutations (preferably substitutions, insertions, or deletions) as compared to the amino acid sequence of any of SEQ ID NO: 1-14;
(b) HCDR2 of the heavy chain variable region is selected from any amino acid sequence of any of SEQ ID NO: 15-28, 121, or a sequence having at least 80%, 85%, 90%, 91%, 92%, 93%, 94%, 95%, 96%, 97%, 98%, 99% or more identity to the amino acid sequence of any of SEQ ID NO: 15-28, 121, or an amino acid sequence having one or more (preferably 2 or 3) conserved amino acid mutations (preferably substitutions, insertions, or deletions) as compared to the amino acid sequence of any of SEQ ID NO: 15-28, 121;
(c) HCDR3 of the heavy chain variable region is selected from any amino acid sequence of any of SEQ ID NO: 29-41, or a sequence having at least 80%, 85%, 90%, 91%, 92%, 93%, 94%, 95%, 96%, 97%, 98%, 99% or more identity to the amino acid sequence of any of SEQ ID NO: 29-41, or an amino acid sequence having one or more (preferably 2 or 3) conserved amino acid mutations (preferably substitutions, insertions, or deletions) as compared to the amino acid sequence of any of SEQ ID NO: 29-41;
(d) LCDR1 of the light chain variable region is selected from any amino acid sequence of SEQ ID NO: 42-54, or a sequence having at least 80%, 85%, 90%, 91%, 92%, 93%, 94%, 95%, 96%, 97%, 98%, 99% or more identity to any amino acid sequence of SEQ ID NO: 42-54, or an amino acid sequence having one or more (preferably 2 or 3) conserved amino acid mutations (preferably substitutions, insertions, or deletions) in comparison to any amino acid sequence of SEQ ID NO: 42-54;
(e) LCDR2 of the light chain variable region is selected from any amino acid sequence of SEQ ID NO: 55-65, or a sequence having at least 80%, 85%, 90%, 91%, 92%, 93%, 94%, 95%, 96%, 97%, 98%, 99% or more identity to any amino acid sequence of SEQ ID NO: 55-65, or an amino acid sequence having one or more (preferably 2 or 3) conserved amino acid mutations (preferably substitutions, insertions, or deletions) in comparison to any amino acid sequence of SEQ ID NO: 55-65; and/or
(f) LCDR3 of the light chain variable region is selected from any amino acid sequence of SEQ ID NO: 66-79, or a sequence having at least 80%, 85%, 90%, 91%, 92%, 93%, 94%, 95%, 96%, 97%, 98%, 99% or more identity to any amino acid sequence of SEQ ID NO: 66-79, or an amino acid sequence having one or more (preferably 2 or 3) conserved amino acid mutations (preferably substitutions, insertions, or deletions) in comparison to any amino acid sequence of SEQ ID NO: 66-79.

In some embodiments, HCDR1, HCDR2, HCDR3 of the heavy chain variable region, and LCDR1, LCDR2, LCDR3 of the light chain variable region are selected from the amino acid sequences of any of the following (1)-(17):
(1) HCDR1 as shown in SEQ ID NO: 1, HCDR2 as shown in SEQ ID NO: 15, HCDR3 as shown in SEQ ID NO: 29, LCDR1 as shown in SEQ ID NO: 42, LCDR2 as shown in SEQ ID NO: 55, and LCDR3 as shown in SEQ ID NO: 66;
(2) HCDR1 as shown in SEQ ID NO: 2, HCDR2 as shown in SEQ ID NO: 16, HCDR3 as shown in SEQ ID NO: 30, LCDR1 as shown in SEQ ID NO: 43, LCDR2 as shown in SEQ ID NO: 56, and LCDR3 as shown in SEQ ID NO: 67;
(3) HCDR1 as shown in SEQ ID NO: 3, HCDR2 as shown in SEQ ID NO: 17, HCDR3 as shown in SEQ ID NO: 31, LCDR1 as shown in SEQ ID NO: 44, LCDR2 as shown in SEQ ID NO: 57, and LCDR3 as shown in SEQ ID NO: 68;
(4) HCDR1 as shown in SEQ ID NO: 4, HCDR2 as shown in SEQ ID NO: 18, HCDR3 as shown in SEQ ID NO: 32, LCDR1 as shown in SEQ ID NO: 45, LCDR2 as shown in SEQ ID NO: 58, and LCDR3 as shown in SEQ ID NO: 69;
(5) HCDR1 as shown in SEQ ID NO: 5, HCDR2 as shown in SEQ ID NO: 19, HCDR3 as shown in SEQ ID NO: 33, LCDR1 as shown in SEQ ID NO: 46, LCDR2 as shown in SEQ ID NO: 59, and LCDR3 as shown in SEQ ID NO: 70;
(6) HCDR1 as shown in SEQ ID NO: 6, HCDR2 as shown in SEQ ID NO: 20, HCDR3 as shown in SEQ ID NO: 34, LCDR1 as shown in SEQ ID NO: 47, LCDR2 as shown in SEQ ID NO: 60, and LCDR3 as shown in SEQ ID NO: 67;
(7) HCDR1 as shown in SEQ ID NO: 6, HCDR2 as shown in SEQ ID NO: 20, HCDR3 as shown in SEQ ID NO: 34, LCDR1 as shown in SEQ ID NO: 48, LCDR2 as shown in SEQ ID NO: 61, and LCDR3 as shown in SEQ ID NO: 71;
(8) HCDR1 as shown in SEQ ID NO: 7, HCDR2 as shown in SEQ ID NO: 21, HCDR3 as shown in SEQ ID NO: 35, LCDR1 as shown in SEQ ID NO: 49, LCDR2 as shown in SEQ ID NO: 62, and LCDR3 as shown in SEQ ID NO: 72;
(9) HCDR1 as shown in SEQ ID NO: 8, HCDR2 as shown in SEQ ID NO: 22, HCDR3 as shown in SEQ ID NO: 36, LCDR1 as shown in SEQ ID NO: 50, LCDR2 as shown in SEQ ID NO: 63, and LCDR3 as shown in SEQ ID NO: 73;
(10) HCDR1 as shown in SEQ ID NO: 9, HCDR2 as shown in SEQ ID NO: 23, HCDR3 as shown in SEQ ID NO: 30, LCDR1 as shown in SEQ ID NO: 51, LCDR2 as shown in SEQ ID NO: 56, and LCDR3 as shown in SEQ ID NO: 74;
(11) HCDR1 as shown in SEQ ID NO: 10, HCDR2 as shown in SEQ ID NO: 24, HCDR3 as shown in SEQ ID NO: 37, LCDR1 as shown in SEQ ID NO: 50, LCDR2 as shown in SEQ ID NO: 63, and LCDR3 as shown in SEQ ID NO: 75;
(12) HCDR1 as shown in SEQ ID NO: 11, HCDR2 as shown in SEQ ID NO: 24, HCDR3 as shown in SEQ ID NO: 37, LCDR1 as shown in SEQ ID NO: 50, LCDR2 as shown in SEQ ID NO: 63, and LCDR3 as shown in SEQ ID NO: 76;
(13) HCDR1 as shown in SEQ ID NO: 12, HCDR2 as shown in SEQ ID NO: 25, HCDR3 as shown in SEQ ID NO: 38, LCDR1 as shown in SEQ ID NO: 52, LCDR2 as shown in SEQ ID NO: 64, and LCDR3 as shown in SEQ ID NO: 67;
(14) HCDR1 as shown in SEQ ID NO: 2, HCDR2 as shown in SEQ ID NO: 26, HCDR3 as shown in SEQ ID NO: 39, LCDR1 as shown in SEQ ID NO: 51, LCDR2 as shown in SEQ ID NO: 56, and LCDR3 as shown in SEQ ID NO: 77;
(15) HCDR1 as shown in SEQ ID NO: 13, HCDR2 as shown in SEQ ID NO: 27, HCDR3 as shown in SEQ ID NO: 40, LCDR1 as shown in SEQ ID NO: 53, LCDR2 as shown in SEQ ID NO: 57, and LCDR3 as shown in SEQ ID NO: 78;
(16) HCDR1 as shown in SEQ ID NO: 14, HCDR2 as shown in SEQ ID NO: 28, HCDR3 as shown in SEQ ID NO: 41, LCDR1 as shown in SEQ ID NO: 54, LCDR2 as shown in SEQ ID NO: 65, and LCDR3 as shown in SEQ ID NO: 79; and
(17) HCDR1 as shown in SEQ ID NO: 2, HCDR2 as shown in SEQ ID NO: 121, HCDR3 as shown in SEQ ID NO: 30, LCDR1 as shown in SEQ ID NO: 43, LCDR2 as shown in SEQ ID NO: 56, and LCDR3 as shown in SEQ ID NO: 67.

In some embodiments, the antibody against CEACAM5 and CEACAM6 or antigen-binding fragment thereof comprises a heavy chain variable region and a light chain variable region, wherein,
(a) the heavy chain variable region has an amino acid sequence of any of the amino acids that are given in SEQ ID NO: 80-94, 122-125, or a sequence having at least 80%, 85%, 90%, 91%, 92%, 93%, 94%, 95%, 96%, 97%, 98%, 99% or more identity to an amino acid sequence that are given in SEQ ID NO: 80-94, 122-125 or an amino acid sequence having one or more (preferably 1, 2, 3, 4, 5, 6, 7, 8, 9, 10) conserved amino acid mutations (preferably substitutions, insertions, or deletions) in comparison to any of the amino acid sequences of SEQ ID NO: 80-94, 122-125;
(b) the light chain variable region has an amino acid sequence of any of the amino acids that are given in SEQ ID NO: 95-110, 126-128, or a sequence having at least 80%, 85%, 90%, 91%, 92%, 93%, 94%, 95%, 96%, 97%, 98%, 99% or more identity to an amino acid sequence that are given in SEQ ID NO: 95-110, 126-128, or an amino acid sequence having one or more (preferably 1, 2, 3, 4, 5, 6, 7, 8, 9, 10) conserved amino acid mutations (preferably substitutions, insertions, or deletions) in comparison to any of the amino acid sequences of SEQ ID NO: 95-110, 126-128.

In some embodiments, the heavy chain variable region and the light chain variable region are selected from any of the following amino acid sequences (1)-(28):
(1) SEQ ID NO: 80 and SEQ ID NO: 95;
(2) SEQ ID NO: 81 and SEQ ID NO: 96;
(3) SEQ ID NO: 82 and SEQ ID NO: 97;
(4) SEQ ID NO: 83 and SEQ ID NO: 98;
(5) SEQ ID NO: 84 and SEQ ID NO: 99;
(6) SEQ ID NO: 85 and SEQ ID NO: 100;
(7) SEQ ID NO: 85 and SEQ ID NO: 101;
(8) SEQ ID NO: 86 and SEQ ID NO: 102;
(9) SEQ ID NO: 87 and SEQ ID NO: 103;
(10) SEQ ID NO: 88 and SEQ ID NO: 104;
(11) SEQ ID NO: 89 and SEQ ID NO: 105;
(12) SEQ ID NO: 90 and SEQ ID NO: 106;
(13) SEQ ID NO: 91 and SEQ ID NO: 107;
(14) SEQ ID NO: 92 and SEQ ID NO: 108;
(15) SEQ ID NO: 93 and SEQ ID NO: 109;
(16) SEQ ID NO: 94 and SEQ ID NO: 110;
(17) SEQ ID NO: 122 and SEQ ID NO: 126;
(18) SEQ ID NO: 122 and SEQ ID NO: 127;
(19) SEQ ID NO: 122 and SEQ ID NO: 128;
(20) SEQ ID NO: 123 and SEQ ID NO: 126;
(21) SEQ ID NO: 123 and SEQ ID NO: 127;
(22) SEQ ID NO: 123 and SEQ ID NO: 128;
(23) SEQ ID NO: 124 and SEQ ID NO: 126;
(24) SEQ ID NO: 124 and SEQ ID NO: 127;
(25) SEQ ID NO: 124 and SEQ ID NO: 128;
(26) SEQ ID NO: 125 and SEQ ID NO: 126;
(27) SEQ ID NO: 125 and SEQ ID NO: 127; and
(28) SEQ ID NO: 125 and SEQ ID NO: 128.

In some embodiments, the antibody is a mouse-derived antibody, a chimeric antibody, a humanized antibody or a fully human antibody.

In some embodiments, the antibody is a monoclonal antibody.

In some embodiments, the antibody against CEACAM5 and CEACAM6 or antigen-binding fragment further comprises an Fc region, and the Fc region is selected from the group consisting of: mouse IgG1, IgG2a, IgG2b and/or IgG3, or rat IgG1, IgG2a, IgG2b and/or IgG2c.

In some embodiments, the antibody against CEACAM5 and CEACAM6 or antigen-binding fragment thereof further comprises an Fc region, and the Fc region is selected from the group consisting of: human IgG1, IgG2, IgG3 and/or IgG4 or an amino acid sequence in the Fc region with one or more amino acid mutations (preferably substitutions, insertions or deletions) with human IgG1, IgG2, IgG3, IgG4.

The present invention also provides a nucleic acid molecule encoding the antibody against CEACAM5 and CEACAM6 or antigen-binding fragment thereof as described in any one of the above.

The present invention also provides a multifunctional fusion protein comprising the antibody against CEACAM5 and CEACAM6 or antigen-binding fragment thereof as described in any one of the above.

In some embodiments, the multifunctional fusion protein further comprises one or more third antibodies or antigen-binding portions thereof that bind specifically to other antigens.

In some embodiments, the antigen binding the third antibody or antigen-binding portion thereof is selected from the group consisting of: a tumor-associated antigen (TAA) or an immune checkpoint.

In some embodiments, the immune checkpoint is PD-L1, CTLA4, PD-L2, PD-1, 4-1BB, CD47, TIGIT, GITR, TIM3, ILT4, TNFR2, TREM2, LAG3, CD27, B7H3 or B7H4.

In some embodiments, the multifunctional fusion protein further comprises a cytokine.

In some embodiments, the cytokine is selected from the group consisting of: IL-1, IL-2, IL-2 Rα, IL-2 Rβ, IL-3, IL-3 Rα, IL-4, IL-4 Rα, IL-5, IL-5 Rα, IL-6, IL-6 Rα, IL-7, IL-7 Rα, IL-8, IL-9, IL-9 Rα, IL-10, IL-10R1, IL-10R2, IL-11, IL-11 Rα, IL-12, IL-12 Rα, IL-12 Rβ2, IL-12 Rβ1, IL-13, IL-13 Rα, IL-13 Rα2, IL-14, IL-15, IL-15Rα sushi, IL-16, IL-17, IL-18, IL-19, IL-20, IL-20R1, IL-20R2, IL-21, IL-21 Rα, IL-22, IL-23, IL-23R, IL-27 R, IL-31 R, TGF, VEGF, IFNγ, IFNα or GM-CSF.

The use of the antibody against CEACAM5 and CEACAM6 or antigen-binding fragments thereof as described in any one of the above, or multifunctional fusion proteins as described in any one of the above, in the preparation of a drug for the treatment of cancer.

In some embodiments, the cancer is medullary thyroid cancer (MTC), colorectal cancer, hepatocellular cancer, stomach cancer, esophageal cancer, lung cancer, breast cancer, pancreatic cancer, ovarian cancer, uterine cancer, cervical cancer, endometrial cancer, head and neck cancer, bladder cancer, urogenital epithelial cancer, prostate cancer, non-small cell lung cancer, hematopoietic cancer, leukemia and melanoma.

In some embodiments, the use is achieved by one or more of tumor immunotherapy, cell therapy, and gene therapy.

The present invention also provides a pharmaceutical composition comprising an antibody against CEACAM5 and CEACAM6 or antigen-binding fragment thereof as described in any one of the above and a pharmaceutically acceptable carrier, diluent or excipient.

The present invention also provides a pharmaceutical composition comprising the multifunctional fusion protein described in any one of the above and a pharmaceutically acceptable carrier, diluent or excipient.

The present invention also provides use of the antibody against CEACAM5 and CEACAM6 or antigen-binding fragments thereof described in any of the above in the preparation of a CEACAM-5 antigen detection kit.

The present invention also provides use of the antibody against CEACAM5 and CEACAM6 or antigen-binding fragment thereof described in any one of the above in the preparation of a CEACAM-6 antigen detection kit.

The present invention also provides an antibody-drug conjugate compound comprising the antibody against CEACAM5 and CEACAM6 or antigen-binding fragment thereof as described in any one of the above.

In some embodiments, the conjugate is selected from the group consisting of: a cytotoxin, a small molecule chemical, or an immunotoxin.

### Abbreviations and Definitions of Terms

mAb: monoclonal antibody

VH: antibody heavy chain variable region

VL: antibody light chain variable region

CDR: complementary determining regions in the variable region of immunoglobulins

FR: antibody framework region, i.e., amino acid residues other than CDR residues in the variable region of the antibody

IgG: immunoglobulin G.

The term “antibody” refers to a natural immunoglobulin or an immunoglobulin prepared by partial or complete synthesis. The antibody may be isolated from a natural source such as plasma or serum in which the antibody is naturally present, or from the culture supernatant of antibody-producing hybridoma cells, or from the immune serum of an animal, or from reconstruction by phage library screening. Alternatively, they may be partially or completely synthesized by using techniques such as genetic recombination. Preferred antibodies include, for example, antibodies to isoforms of immunoglobulins or subclasses of these isoforms. Human immunoglobulins are known to include nine classes (isotypes) of IgG1, IgG2, IgG3, IgG4, IgA1, IgA2, IgD, IgE, and IgM. Within these isotypes, the antibodies of the present invention may include IgG1, IgG2, IgG3, and/or IgG4.

The term “monoclonal antibody” refers to a homogeneous antibody directed only to a specific antigenic epitope. In contrast to common polyclonal antibody preparations, which typically include different antibodies directed against different antigenic determinants (epitopes), each monoclonal antibody is directed against a single antigenic determinant on the antigen. The modifier “monoclonal” denotes a homogeneous characterization of the antibody and is not to be construed as requiring that the antibody be produced by any particular method. The monoclonal antibodies of the present invention are preferably produced by recombinant DNA methods or obtained by screening methods described elsewhere herein.

The term “mouse-derived antibody” is used in the present invention for monoclonal antibodies prepared according to the knowledge and skill in the art. The preparation involves injecting a test subject with an antigen and then isolating a hybridoma expressing an antibody having the desired sequence or functional properties.

The term “chimeric antibody” is used to describe an antibody made by fusing the variable region of a mouse-derived antibody with the constant region of a human antibody, which can reduce the immune response induced by the mouse-derived antibody. To establish a chimeric antibody, a hybridoma secreting a mouse-specific monoclonal antibody is established, the variable region gene is cloned from the mouse hybridoma cells, the constant region gene of the human antibody is cloned according to the need, and the chimeric gene is inserted into the human vector by linking the mouse variable region gene and the human constant region gene, and then the chimeric antibody molecule is expressed in the eukaryotic or prokaryotic industrial system.

The term “humanized antibody”, also known as CDR-transplanted antibody, refers to antibodies produced by transplanting mouse CDR sequences into the variable region framework of human antibodies, i.e., different types of human germline antibody framework sequences. It can overcome the strong heterologous response induced by chimeric antibodies due to carrying a large number of mouse protein components.

Some of the antibodies used herein consist of an immunoglobulin molecule comprising two pairs of polypeptide chains, each pair having a light chain (LC) and a heavy chain (HC). Each heavy chain consists of a heavy chain variable region (VH) and a heavy chain constant region (CH). The heavy chain constant region consists of three structural domains (CH1, CH2 and CH3). Each light chain consists of a light chain variable region (VL) and a light chain constant region (CL), or only a light chain constant region (CL). The light chain constant region consists of a structural domain CL. The constant structural domain is not directly involved in antibody-antigen binding, but exhibits a variety of effector functions, such as the ability to mediate the binding of immunoglobulins to host tissues or factors, including various cells of the immune system (e.g., effector cells) and the first component of the classical complement system (C1q). The VH and VL regions can also be subdivided into regions with high variability (called complementary determining regions (CDRs)), with more conserved regions called framing regions (FRs) scattered in between. Each VH and VL consists of three CDRs and four FRs arranged from amino-terminal to carboxy-terminal in the following order: FR1, CDR1, FR2, CDR2, FR3, CDR3, FR4. The variable regions of each heavy chain/light chain pair (VH and VL) form the antigen-binding site, respectively.

The term “antigen-binding fragment” of an antibody refers to a polypeptide fragment of an antibody, such as a polypeptide fragment of a full-length antibody, which maintains the ability to specifically bind the same antigen bound by the full-length antibody and/or to compete with the full-length antibody for specific binding of the antigen, and which is also referred to as an “antigen-binding portion”. The antigen-binding portion of an antibody may be produced by recombinant DNA technology or by enzymatic or chemical breakage of the intact antibody. Non-limiting examples of antigen-binding fragments include Fab, Fab′, F(ab′)2, Fd, Fv, dAb and complementary determining region (CDR) fragments, single chain antibodies (e.g., scFv), chimeric antibodies, diabodies, linear antibodies, nanobodies (e.g., technology from Ablynx), structural domain antibodies (e.g., technology from Domantis), and such peptides comprising at least a portion of an antibody sufficient to confer specific antigen-binding ability to the peptide.

The term “antibody-drug conjugate” or “ADC” refers to a binding protein (e.g., an antibody or antigen-binding fragment thereof) that is coupled to one or more coupled drugs (which may optionally be therapeutic or cytotoxic agents), and whose structure typically consists of three parts: the antibody or antibody-like ligand, the drug portion, and a linker that couples the antibody or antibody-like ligand and the drug. An ADC typically has 1, 2, 3, 4, 5, 6, 7, 8, 9, or 10 numbers of drugs coupled to the antibody.

The term “polypeptide” refers to a chain of amino acids of any length, irrespective of modification (e.g. phosphorylation or glycosylation). The term polypeptide includes proteins and fragments thereof. Polypeptides may be “exogenous”, meaning that they are “heterologous”, i.e., foreign to the host cell being utilized, e.g., human polypeptides produced by bacterial cells. Polypeptides are disclosed herein as sequences of amino acid residues. Those sequences are written from left to right in the direction from the amino terminus to the carboxy terminus. According to standard nomenclature, amino acid residue sequences are named in three-letter or one-letter codes as follows: Alanine (Ala, A), Arginine (Arg, R), Asparagine (Asn, N), Aspartic acid (Asp, D), Cysteine (Cys, C), Glutamine (Gln, Q), Glutamic acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y) and Valine (Val, V).

The term “host cell” refers to a cell that has been or is capable of being transformed with a nucleic acid sequence and thus expressing the selected target gene. The term includes the progeny of the parental cell, whether or not the progeny is morphologically or genetically identical to the original parental cell, as long as the selected target gene is present in the progeny. Commonly used host cells include bacteria, yeast, mammalian cells, etc.

The term “vector” refers to a nucleic acid molecule capable of proliferating another nucleic acid to which it is attached. The term includes vectors that are self-replicating nucleic acid structures and vectors that are incorporated into the genome of the host cell into which they are introduced. Certain vectors are capable of directing the expression of nucleic acids operably linked to them. Such vectors are referred to herein as “expression vectors”.

The term “pharmaceutically acceptable carrier” includes any standard drug carrier, such as phosphate buffered saline solutions, water and emulsions, as well as various types of wetting agents.

The term “identity” is defined as the percentage of amino acid residues in a candidate sequence that are identical to amino acid residues in a control polypeptide sequence after comparing the sequences and introducing gaps where necessary to obtain the maximum percentage sequence identity. Comparisons for the purpose of determining percentage amino acid sequence identity can be performed in a variety of ways within the skill of the art, for example, using publicly available computer software, such as BLAST software or the FASTA package.

There are a variety of methods/systems in the field to define and characterize CDRs that have been developed and refined over the years, including Kabat, Chothia, IMGT, AbM, and Contact; Kabat is the most commonly used and defines CDRs based on sequence variability; Chothia defines CDRs based on sequence variability based on the position of the cyclic region of the structure; the IMGT system defines CDRs based on sequence variability and position within the variable domain structure; AbM is defined based on Oxford Molecules' AbM antibody modeling software and is a compromise between Kabat and Chothia; Contact defines CDRs based on the analysis of complex crystal structures, and is similar to Chothia in several respects.

The CDRs of the anti-CEACAM5 and CEACAM6 antibodies of the present invention refer to highly variable regions of the heavy and light chains of the immunoglobulins, wherein the CDR amino acid positions of SEQ ID NO: 1-79 are defined in accordance with Kabat and the CDR amino acid positions of SEQ ID NO: 121 are defined in accordance with AbM.

The term “specificity” refers to the fact that one of the molecules involved in specific binding does not show any significant binding to a molecule or molecules different from one or more of the binding partner molecules. In addition, the term is used when the structural domain containing the variable region of the antibody is specific to a particular epitope of a plurality of epitopes in the antigen. When the epitope to which the domain containing the antibody variable region binds is contained in several different antigens, an antigen-binding molecule comprising the domain containing the antibody variable region can bind a variety of antigens having said epitopes.

The term “epitope” refers to an antigenic determinant cluster in an antigen and the antigenic site bound by the structural domain of an antigen-binding molecule comprising an antibody variable region as disclosed in this specification. Thus, the epitope can be defined based on its structure. In addition, the epitope may also be defined based on the antigen-binding activity in the antigen-binding molecule that recognizes the epitope. When the antigen is a peptide or polypeptide, the epitope may be designated by the amino acid residues forming the epitope. When the epitope is a glycan chain, the epitope may be defined by its specific glycan chain structure.

The term “positive control antibody” refers to a natural or engineered cell that binds or expresses a target protein.

The term “isotype control” refers to a negative control that is used in the same experiment as the experimental sample of the same genus, same subtype, same dose, same immunoglobulin and immunoglobulin of subtype, same labeling, etc., and is used to eliminate the experimental background effect of the non-specifically bound sample on the experimental values in the experiment, and as a more illustrative control of the effect of the experiment.

The anti-CEACAM5 and CEACAM6 antibodies provided by the present invention are capable of differentially binding to tumor tissues and normal tissues, reducing the adverse effects of non-specific binding. The antibodies provided by the present invention also have good endocytosis activity and can be used for anti-tumor therapy of coupled small molecule drugs forming ADC drugs.

### BRIEF DESCRIPTION OF THE DRAWINGS

FIG. 1 shows the binding activity of mouse-derived antibodies 1, 2, 3, 4, and 5 to HPAFII cells.

FIG. 2 shows the binding activity of mouse-derived antibodies 6, 7, and 8 to HPAFII cells.

FIG. 3 shows the binding activity of mouse-derived antibodies 9, 10, and 11 to HPAFII cells.

FIG. 4 shows the binding activity of mouse-derived antibodies 12, 13, 14, 15 and 16 to HPAFII cells.

FIG. 5 shows the binding activity of mouse-derived antibodies 1-9 to human CEACAM6 as detected by ELSIA.

FIG. 6 shows the binding activity of mouse-derived antibodies 9-16 to human CEACAM6 as detected by ELSIA.

FIG. 7 shows the binding activity of mouse-derived antibodies 1-8 to human CEACAM5 as detected by ELSIA.

FIG. 8 shows the binding activity of mouse-derived antibodies 9-15 to human CEACAM5 as detected by ELSIA.

FIG. 9 shows the binding activity of mouse-derived antibodies 1-8 to human CEACAM6 (AB) as detected by ELSIA.

FIG. 10 shows the binding activity of mouse-derived antibodies 9-15 to human CEACAM6 (AB) as detected by ELSIA.

FIG. 11 shows the results of binding activity of chimeric antibodies, humanized antibodies and HPAFII by FACS assay.

FIG. 12 shows the ADCC activity of the chimeric antibody against the target cells.

FIG. 13 shows the survival effect (internalization) of antibody Fab ZAP conjugate on HPAFII cells.

### DETAILED DESCRIPTION

The present invention is further described below in connection with the accompanying drawings and specific embodiments, the protection of which is not limited to the following embodiments. It should also be understood that the terms used in embodiments of the present invention are intended to describe particular specific embodiments and are not intended to limit the scope of protection of the present invention. Without departing from the spirit and scope of the inventive concept, variations and advantages that can be envisaged by those skilled in the art are included in the present invention and the scope of protection of the invention by the appended claims and any equivalents thereof.

### Example 1 Animal Immunization

Balb/c mice were immunized with human CEACAM5-his (ACRO CE5-H5226, protein number UniProtKB—P06731) and human CEACAM6-his (ACRO CE6-H5223, protein number UniProtKB—P40199) proteins as immunogens, mixed with Freund's adjuvant. A secondary immunization was performed 2 weeks after the first immunization and then again three weeks later. Mouses were taken negative serum 3 days before immunization, and 50 μL of blood was taken by tail clipping 6 days after each immunization. Negative and immune sera were diluted proportionally (1:0.1K, 1:1K, 1:10K, 1:100K, 1:1000K, 1:10,000K), and serum titer was detected with overexpressed human CEACAM5-his and human CEACAM6-his proteins by ELISA. When the titer result meets the requirement and anti-human CEACAM5 and CEACAM6 antibody is detected at a dilution of >1:10K, rat spleen and lymph nodes can be harvested.

### Example 2 Cell Fusion

B lymphocytes and lymph node cells for experiments were obtained from Balb/c mouse immunized four times, and the spleen and lymph nodes were placed in a cell sieve, which was then placed in a 50 mL centrifuge tube. DMEM was pipetted dropwise onto spleens for grinding to make a suspension of splenocytes, and centrifuged, at 1600 rpm, for 10 min, and supernatants were removed. B cells were resuspended with 2 mL of erythrocyte lysate, and lysed for 2 minutes at room temperature, then 30 mL of DMEM was added, and mixed and centrifuged at 1600 rpm for 10 minutes, and counted.

Myeloma cells SP2/0 (ATCC) were passaged one day before fusion so that the cells were in the logarithmic growth phase at the time of the experiment. Splenocytes and SP2/0 were mixed in a 2:1 ratio and centrifuged at 1600 rpm for 10 min. The mixed cells were washed twice with fusion solution and centrifuged at 1600 rpm for 10 minutes. Cells were suspended by adding fusion solution at a final cell density of 1×10⁷ cells/mL, and within 5 minutes, the cell suspension was transferred to the fusion chamber of an electrofusion apparatus (BTX; ECM 2001) for fusion. After completion of fusion the cells were moved from the fusion chamber to complete medium containing HAT and incubated at 37° C. for 60 min. After incubation the cells were spread in 96-well plates already containing feeder cells and incubated at 37° C. with 5% CO₂.

### Example 3 ELISA Method to Screen Positive Clones

Primary screening of fusion supernatants was performed after 7 days of incubation. Human-CEACAM5-His (ACRO CE5-H5226, protein number UniProtKB—P06731), Human-CEACAM6-His (ACRO CE6-H5223, Protein No. UniProtKB—P40199), Human-CEACAM6 (AB)-His (Sequence SEQ ID NO: 138, with 6 His tags added at the C-terminal end, obtained by purification through nickel column) were diluted to 1 μg/mL using PBS buffer at pH 7.4, and 100 μL per well was added to 96-well ELISA plates, and coated at 4° C. overnight. After blocked with 1% BSA blocking solution for 1 h, and then the plate was washed 3 times with PBST, then the constructed antibody was diluted with 0.5% BSA sample diluent to 10 μg/mL, and this was used as the initial concentration, and 3-fold gradient dilution was carried out, with a total of 11 gradients, and 100 μL per well, and incubated at 37° C. for 1 h. The plate was was washed 3 times with PBST, and HRP-labeled goat anti-human IgGFc was diluted with sample diluent at 1:20,000, and 100 μL was added to each well, and incubated at room temperature for 1 h.

Negative control (blank wells and IgG1 isotype control) and positive control were set up, and the positive control was NEO-201 antibody (msIgG2a subtype, see CN 111670199 A) that can bind CEACAM5, CEACAM6, CEACAM6 (AB) simultaneously, and the variable region of the positive control antibody sequence consisted of SEQ ID No. 119, SEQ ID No. 120, which linked the constant region of mouse IgG2a. The plate was washed 4 times with PBST, 100 μL of TMB substrate was added to each well, and incubated at room temperature for 10 minutes protected from light, and the color development reaction was terminated by adding 100 μL of 1M HCl solution to each well. The absorbance value of each well in the 96-well plate was determined by selecting the wavelength of 450 nm and the reference wavelength of 570 nm on a multifunctional enzyme-labeled instrument, the absorbance value of each well (OD)=OD450 nm-OD570 nm, and the data were analyzed. The cell lines whose supernatants reacted with the three encapsulated proteins with the result of OD>1.0 were selected as the candidate positive cell lines for primary screening, and the culture supernatants of the positive cell lines were aspirated, discarded, and new HAT complete medium was added.

### Example 4 FACS Method to Screen Positive Clones for Binding to Tumor Cells

HCC827 cells (From USTC SHANGHAI INSTITUTE FOR ADVANCED STUDIES) were taken and transferred to a centrifuge tube and centrifuged at 1000 rpm for 5 min. A 100 μL aliquot of 3×10⁵ stably expressing cells was divided into separate tubes and 100 μL of fusion supernatant was added. The cells were incubated at 4° C. for 60 min, and then washed twice with excess FACS buffer. Cells were resuspended in 100 μL FACS buffer, and sheep anti-mouse secondary antibody-FITC (Abcam; ab6785) was added to the samples, then incubated for 30 minutes and washed twice with excess FACS buffer. Cells were fixated in fixation buffer and subsequently analyzed by flow cytometry. The FACS method was used to screen for antibodies that bind specifically to HCC827 cells.

Two rounds of limited dilution were used for monocloning of hybridoma cells, which were detected by ELISA, and single clones with OD450>1.0 were selected as definitive candidate cell lines for passaging, and clones without monoclonal antibody were selected for the next subcloning with OD450>1.0.

### Example 5 Antibody Sample Production for Candidate Cell Lines

Hybridoma cells were cultured in T75 cell culture flasks until cell coverage was 80-90%. Discard the cell supernatant from 2 bottles, then 30 mL hybridoma-SFM was added and cultured at 37° C. with 5% CO₂. Cultivate for 2-3 days, the cell status and medium color were observed, if the medium color turned yellow, then 30 mL of new hybridoma-SFM could be added. It was cultured for 6-7 days, the culture supernatant was collected by low-speed centrifugation, and purification was carried out.

### Example 6 Binding Activity of Candidate Antibodies to HPAFII Cells

The antibody to be tested was taken as the initial concentration of 200 nM and diluted 5-fold with 6 gradients. The HPAFII cells (From USTC SHANGHAI INSTITUTE FOR ADVANCED STUDIES) in the incubator were taken out, and the cell suspension was transferred to a 15 mL centrifuge tube, centrifuged, and counted by resuspension in PBS. A blank control group (Blank), a negative control group (NC), an experimental group, a positive control group and the irrelevant antibody group were kept. Positive control is a NEO-201 antibody (msIgG2a isoform, see CN 111670199 A) that binds to both CEACAM5, CEACAM6, and CEACAM6 (AB), and the variable region of the sequence of the positive control antibody consists of SEQ ID No. 119, SEQ ID No. 120, which joins the constant region of mouse IgG2a. The cell suspension was spread in a 96-well plate according to about 3×10⁵ cells/well. Centrifugation (1000 rpm, 5 min) was performed, followed by washing with PBS, re-centrifugation, and repeated twice to remove media residues. The supernatant was discarded, 100 μL of primary antibody solution and irrelevant antibody solution were added to the experimental group and irrelevant antibody group, respectively, and the cells were resuspended and incubated at room temperature for 1 h. The blank group, and the negative control group were incubated with an equal amount of PBS.

After 1 h, PBS was added to wash twice. After the supernatant was discarded, 100 μL of fluorescent secondary antibody dilution (Mouse-derived secondary antibody from Abcam ab6785) was added to each sample group, except for the blank group which was added with 100 μL of PBS. After incubation at room temperature and protected from light for 0.5 h, centrifugation was performed and PBS added to wash the samples twice. After the supernatant was discarded, 120 μL of PBS was added to resuspend and sequentially flow cytometry was performed to detect the mean fluorescence intensity. The concentration of the antibody was logarithmically taken as the horizontal coordinate, and the Sigmoidaldose-response (VariableSlope) method (GraphPadPrism Software, GraphPadSoftware, San Diego, California) was chosen to perform a nonlinear regression to obtain the binding activity curves of the antibody on HPAFII cells.

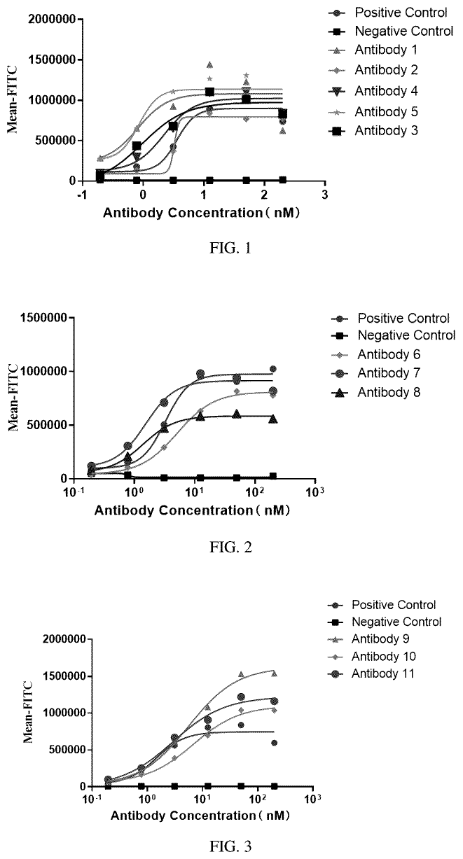

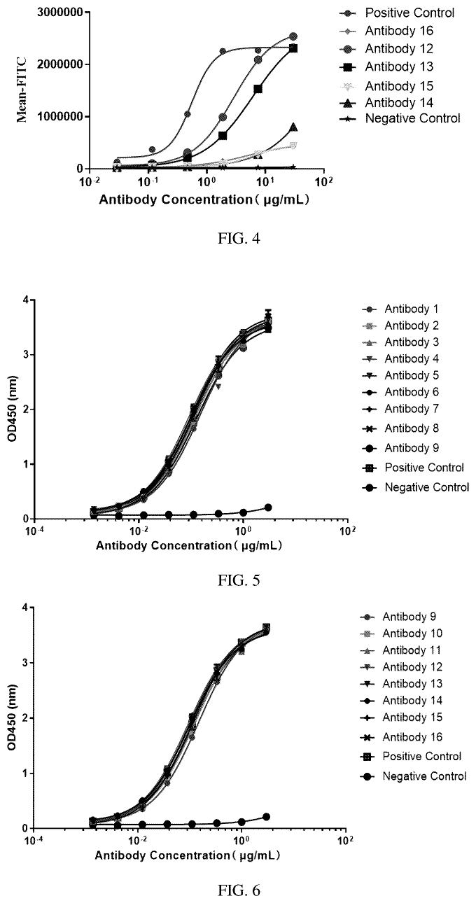

The results are shown in FIGS. 1-4. The results show that the selected mouse-derived antibodies all have good binding activity to HPAFII cells.

### Example 7 Detection of Protein Binding Activity by ELISA Method

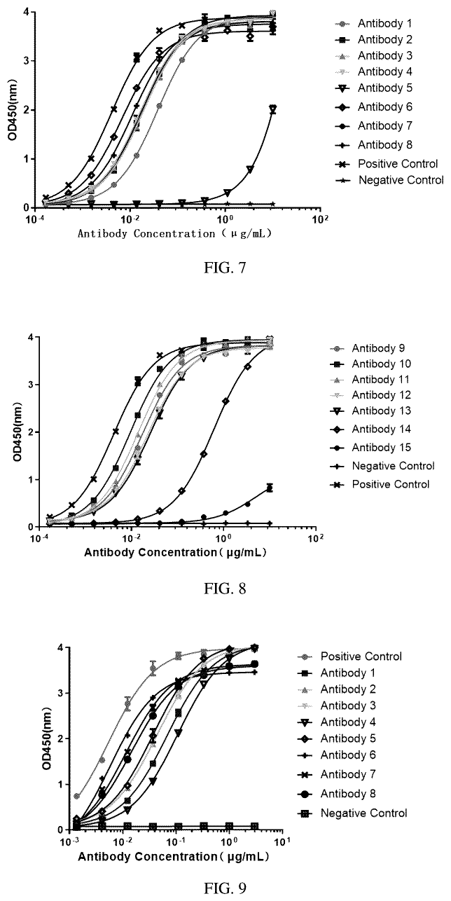

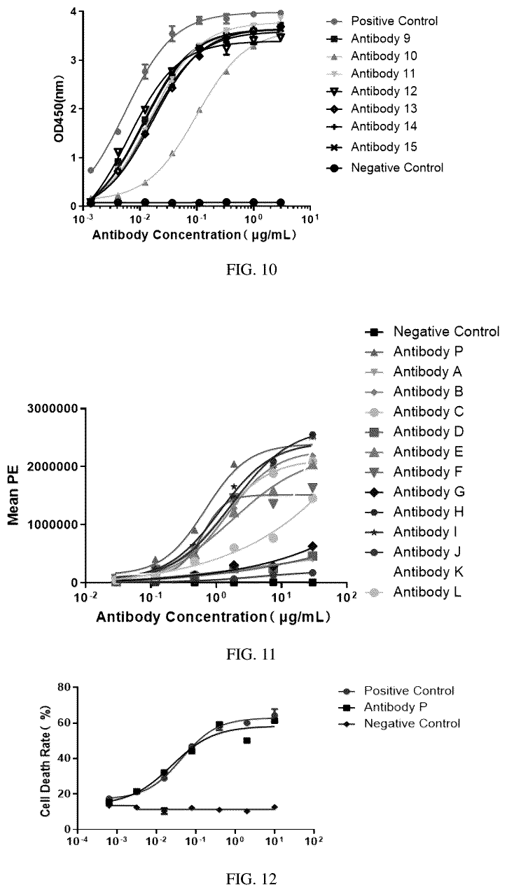

The binding activity of the antibodies to human CEACAM5, human CEACAM6, and human CEACAM6 (AB) proteins was detected by ELISA. The plate coated with proteins was incubated at 4° C. overnight, and the 96-well ELISA plate blocked with 2% PBS-BSA was added with the antibody to be detected and incubated at 37° C. for 1 h. After washing 3 times with PBS containing 0.05% Tween, the absorbance value of each well was read at wavelength 450 nm with the gradient-diluted mouse-derived candidate antibody as the primary antibody and anti-mouse-IgG-FC-HRP as the secondary antibody, and the absorbance value of each well was read at wavelength 450 nm after the TMB color development. The results of detection of antibody binding to human CEACAM5, human CEACAM6 and CEACAM6 (AB) are shown in FIGS. 5-10, respectively, which show that the selected mouse-derived antibodies all bind to the proteins.

### Example 8 Species Cross-Testing by ELISA Methods

The affinity of the antibodies to monkey CEACAM5, monkey CEACAM6, and mouse CEACAM5 proteins was determined by ELISA. The plates encapsulated with human or mouse proteins were incubated at 4° C. overnight, and the antibodies to be detected were added to 96-well ELISA plates after being blocked with 2% PBS-BSA and incubated at 37° C. for 1 h. After washing 3 times with PBS containing 0.05% Tween, the absorbance value of each well was read at wavelength 450 nm with the antibody to be detected as the primary antibody and Goat anti-mouse IgG-FC-HRP as the secondary antibody, and the absorbance value of each well was read at wavelength 450 nm after the TMB color development. The specific binding to CEACAM5 and CEACAM6 of mouse and monkey was detected by Elisa method.

The binding activities of the antibodies to be tested with monkey CEACAM5, monkey CEACAM6, and mouse CEACAM5 proteins are shown in Table 1. The results show that the candidate antibodies bind to monkey CEACAM5 and monkey CEACAM6, and basically do not bind to mouse CEACAM5. That is, the candidate antibody has human and monkey cross-reactivity to CEACAM5 and CEACAM6 proteins.

TABLE 1
Cross-reactivity (OD) of the candidate antibodies with
the species of target proteins of monkey and mouse.

| | Monkey | | Mouse | | Monkey | |
|---|---|---|---|---|---|---|
| Name | CEACAM5 | | CEACAM5 | | CEACAM6 | |
| Antibody 1 | 3.52 | 3.51 | 0.16 | 0.16 | 2.67 | 2.58 |
| Antibody 2 | 3.49 | 3.46 | 0.15 | 0.15 | 3.03 | 2.82 |
| Antibody 3 | 3.40 | 3.27 | 0.10 | 0.09 | 3.08 | 3.11 |
| Antibody 4 | 3.42 | 3.28 | 0.22 | 0.17 | 3.30 | 3.06 |
| Antibody 5 | 3.58 | 3.37 | 0.26 | 0.22 | 3.00 | 2.88 |
| Antibody 6 | 3.74 | 3.61 | 0.45 | 0.39 | 3.53 | 3.18 |
| Antibody 7 | 2.09 | 1.84 | 0.31 | 0.24 | 2.16 | 1.94 |
| Antibody 8 | 3.57 | 3.40 | 0.16 | 0.14 | 2.55 | 2.38 |
| Antibody 9 | 3.38 | 3.43 | 0.14 | 0.13 | 3.16 | 3.18 |
| Antibody 10 | 2.58 | 2.54 | 0.15 | 0.15 | 1.88 | 1.77 |
| Antibody 11 | 3.43 | 3.39 | 0.17 | 0.13 | 3.12 | 3.09 |
| Antibody 12 | 3.54 | 3.51 | 0.15 | 0.14 | 2.84 | 2.85 |
| Antibody 13 | 3.42 | 3.41 | 0.15 | 0.15 | 2.95 | 2.98 |
| Antibody 14 | 3.37 | 3.36 | 0.22 | 0.20 | 2.83 | 2.74 |
| Antibody 15 | 3.43 | 3.31 | 0.16 | 0.14 | 2.89 | 2.92 |
| Antibody 16 | 3.45 | 3.35 | 0.15 | 0.17 | 3.02 | 3.00 |
| BMK (NEO-201) | 3.49 | 3.58 | 0.14 | 0.10 | 3.10 | 2.99 |
| PBS | 0.11 | 0.13 | 0.09 | 0.10 | 0.10 | 0.09 |

### Example 9 Sequencing of Monoclonal Antibodies

The hybridomas with good binding activity to the HPAFII cell obtained by FACs assay screening were sequenced to obtain the heavy chain variable region and light chain variable region of candidate cell line.

TABLE 2
Sequence list of antibodies

| | Heavy Chain Amino | Light Chain Amino |
|---|---|---|
| Name | Acid Sequence | Acid Sequence |
| Antibody 1 | SEQ ID NO: 80 | SEQ ID NO: 95 |
| Antibody 2 | SEQ ID NO: 81 | SEQ ID NO: 96 |
| Antibody 3 | SEQ ID NO: 82 | SEQ ID NO: 97 |
| Antibody 4 | SEQ ID NO: 83 | SEQ ID NO: 98 |
| Antibody 5 | SEQ ID NO: 84 | SEQ ID NO: 99 |
| Antibody 6 | SEQ ID NO: 85 | SEQ ID NO: 100, 101 |
| Antibody 7 | SEQ ID NO: 86 | SEQ ID NO: 102 |
| Antibody 8 | SEQ ID NO: 87 | SEQ ID NO: 103 |
| Antibody 9 | SEQ ID NO: 88 | SEQ ID NO: 104 |
| Antibody 10 | SEQ ID NO: 89 | SEQ ID NO: 105 |
| Antibody 11 | SEQ ID NO: 90 | SEQ ID NO: 106 |
| Antibody 12 | SEQ ID NO: 91 | SEQ ID NO: 107 |
| Antibody 13 | SEQ ID NO: 92 | SEQ ID NO: 108 |
| Antibody 14 | SEQ ID NO: 93 | SEQ ID NO: 109 |
| Antibody 15 | SEQ ID NO: 94 | SEQ ID NO: 110 |

Note:
Two light chains (SEQ ID NO: 100 and SEQ ID NO: 101, respectively) and one heavy chain (SEQ ID NO: 85) were detected by antibody 6.

### Example 10 Engineering Design and Expression of Humanized Sequences

Humanized design of the variable region of mouse-derived antibody 2, the design principle is not to introduce protein modification sites such as glycosylation, deamidation, isomerization, etc., not to introduce integrin-binding sites, cysteine, and the reverse mutation of important amino acids in the framework region should maintain the original physicochemical and biochemical activities. The specific methods are as follows:

The variable region of the mouse-derived antibody 2 was compared with the human germline sequences using the IgBLAST tool, respectively, and the FR was replaced with the human germline sequence with the highest sequence similarity. The sequence of humanized antibody was obtained by selecting IGHV3 major class for the mouse-derived antibody 2 heavy chain design template and IGKV3 major class for light chain design template. And then on the basis of this humanization, several important amino acids affecting the affinity of the antibody were revertively mutated, i.e., mutated to the FR site of the original mouse origin. Percentage of humanization is the similarity ratio of the designed sequence Framework to the Germline sequence Framework, and the designed humanized sequences were compared with the human Germline sequences to select the sequences whose percentage of humanization of the antibody is 90% or more.

The heavy chain CDR2 of the mouse-derived antibody 2 sequence contains a glycosylation site NG, and a set of heavy chains was designed to mutate NG to QG on the heavy chain with the most reverse mutations of mouse-derived sequence. A total of 9 chains were generated in this design, including 5 heavy chains, which were mouse chimeric, fully-human, reverse mutation of 3 sites on the basis of fully-human, reverse mutation of 7 sites on the basis of fully-human, and mutation of NG to QG in CDR2 on the basis of reverse mutation of 7 sites; and 4 light chains, which were mouse chimeric, fully-humanized, reversionary mutation 4 sites on fully-humanized basis, and reverse mutation of 8 sites on fully-humanized basis, and a total of 13 combinations were generated. Wherein, antibody P is a chimeric antibody and antibodies A-L are humanized antibodies, as shown in Table 3.

TABLE 3
Sequences of humanized antibodies

| | Variable Region | Full-length | Variable Region | Full-length |
|---|---|---|---|---|
| Name of | Sequence of | Sequence of | Sequence of Light | Sequence of Light |
| Antibody | Heavy Chain | Heavy Chain | Chain | Chain |
| Antibody P | SEQ ID NO: 81 | SEQ ID NO: 136 | SEQ ID NO: 96 | SEQ ID NO: 137 |
| Antibody A | SEQ ID NO: 122 | SEQ ID NO: 129 | SEQ ID NO: 126 | SEQ ID NO: 133 |
| Antibody B | SEQ ID NO: 122 | SEQ ID NO: 129 | SEQ ID NO: 127 | SEQ ID NO: 134 |
| Antibody C | SEQ ID NO: 122 | SEQ ID NO: 129 | SEQ ID NO: 128 | SEQ ID NO: 135 |
| Antibody D | SEQ ID NO: 123 | SEQ ID NO: 130 | SEQ ID NO: 126 | SEQ ID NO: 133 |
| Antibody E | SEQ ID NO: 123 | SEQ ID NO: 130 | SEQ ID NO: 127 | SEQ ID NO: 134 |
| Antibody F | SEQ ID NO: 123 | SEQ ID NO: 130 | SEQ ID NO: 128 | SEQ ID NO: 135 |
| Antibody G | SEQ ID NO: 124 | SEQ ID NO: 131 | SEQ ID NO: 126 | SEQ ID NO: 133 |
| Antibody H | SEQ ID NO: 124 | SEQ ID NO: 131 | SEQ ID NO: 127 | SEQ ID NO: 134 |
| Antibody I | SEQ ID NO: 124 | SEQ ID NO: 131 | SEQ ID NO: 128 | SEQ ID NO: 135 |
| Antibody J | SEQ ID NO: 125 | SEQ ID NO: 132 | SEQ ID NO: 126 | SEQ ID NO: 133 |
| Antibody K | SEQ ID NO: 125 | SEQ ID NO: 132 | SEQ ID NO: 127 | SEQ ID NO: 134 |
| Antibody L | SEQ ID NO: 125 | SEQ ID NO: 132 | SEQ ID NO: 128 | SEQ ID NO: 135 |

### Example 11 Construction and Expression of Humanized Antibody

The designed antibody sequence was genetically synthesized and constructed into the human IgG framework, and then the antibody fragment was inserted into PCDNA3.1 vector using molecular cloning technology, constructed into a mammalian cell expression plasmid, and then introduced into CHO cells of the host cell line by using liposome transfection, the fermentation supernatant was obtained by using cell Fed-batch, and the fermentation supernatant was taken for affinity chromatography purification to finally purify the constructed humanized antibody.

### Example 12 Binding Activity of Humanized Antibodies to HPAFII Cells

Antibodies to be tested were taken for gradient dilution. The HPAFII cells (From USTC SHANGHAI INSTITUTE FOR ADVANCED STUDIES) in the incubator were taken out, and the cell suspension was transferred to a 15 mL centrifuge tube, centrifuged, and counted by resuspension in PBS. A blank control group (Blank), a negative control group (NC), an experimental group, a positive control group and the irrelevant antibody group were kept. Positive control is a NEO-201 antibody (msIgG2a isoform, see CN 111670199 A) that binds to both CEACAM5, CEACAM6, and CEACAM6 (AB), and the variable region of the sequence of the positive control antibody consists of SEQ ID No. 119, SEQ ID No. 120, which joins the constant region of mouse IgG2a. The cell suspension was spread in a 96-well plate according to about 3×10⁵ cells/well.

Centrifugation (1000 rpm, 5 min) was performed, followed by washing with PBS, re-centrifugation, and repeated twice to remove media residues. The supernatant was discarded, 100 μL of primary antibody solution and irrelevant antibody solution were added to the experimental group and irrelevant antibody group, respectively, and the cells were resuspended and incubated at room temperature for 1 h. The blank group, and the negative control group were incubated with an equal amount of PBS.

After 1 h, PBS was added to wash twice. After the supernatant was discarded, 100 μL of fluorescent secondary antibody dilution (Secondary antibody from Abcam ab98596) was added to each sample group, except for the blank group which was added with 100 μL of PBS. After incubation at room temperature and protected from light for 0.5 h, centrifugation was performed and PBS added to wash the samples twice. After the supernatant was discarded, 120 μL of PBS was added to resuspend and sequentially flow cytometry was performed to detect the mean fluorescence intensity. The concentration of the antibody was logarithmically taken as the horizontal coordinate, and the Sigmoidaldose-response (VariableSlope) method (GraphPadPrism Software, GraphPadSoftware, San Diego, California) was chosen to perform a nonlinear regression to obtain the binding activity curves of the antibody on HPAFII cells.

The results are shown in FIG. 11. The results show that most of the designed sequences of the selected humanized antibodies maintain binding activity to HPAFII cells, similar to chimeric antibodies.

### Example 13 ADCC Activity of Antibody on Target Cells

HCC827 cells were used as target cells, and after centrifugation at 1000 rpm for 4 min at room temperature and resuspension using RPMI1640 basal medium (containing 5% FBS), they were spread in 96-well plates at 1×10⁴ cells/well, and 50 μL/well. The antibody was diluted using RPMI1640 basal medium (containing 5% FBS) at a initial concentration of 10 μg/mL, while a 5-fold gradient of dilutions with 7 concentration gradients of 100 L/well was followed. NK cells were resuspended and added to the corresponding wells at 50 μL/well, and E:T was 3:1. target cell maximum lysis wells (M), target cell spontaneous release wells (ST), effector cell spontaneous release wells (SE), total volume-corrected blank wells (BV), and medium blank control wells (BM) were also set up. After standing for 10 min, centrifuge at 1000 rpm for 4 min at room temperature and incubate in a 5% CO₂, 37° C. CO₂ cell culture incubator for 4 h. Lysate was added to wells M and B-V 45 min ahead of time, mixed well, and centrifuged at 1000 rpm for 4 min at room temperature at the end of incubation. 50 μL of supernatant was aspirated to the LDH analysis plate, then 50 μL/well of substrate dissolved in analysis buffer (assay buffer) was added, and the reaction was carried out for 30 min at room temperature and protected from light, then 50 μL/well of termination solution was added, and it was left to stand for 10 min, and readings were taken at 490 nm to calculate the cell death rate.

$$Cell Death Rate ( \% ) = \frac{{OD value}_{sample well - ST - SE} \times 100}{{OD value}_{M - ST}}$$

The concentration of the constructed antibody was logarithmically taken as the horizontal coordinate, and the Sigmoidaldose-response (VariableSlope) method (GraphPadPrism Software, GraphPadSoftware, SanDiego, California) was chosen to perform a nonlinear regression to obtain the target antibody activity on the ADCC activity profile of the target cells.

As can be seen in FIG. 12, the IgG1 isotype control does not show killing of HCC827 cells, the positive control and candidate antibodies show lysis death of HCC827 cells in a concentration-dependent manner.

### Example 14 Endocytosis Activity

Candidate antibody and Fab-ZAP (Saponin Toxin Protein) formed as a conjugate were co-incubated with tumor cells expressing CEACAM5 and CEACAM6. The antibody binds to CEACAM5 and CEACAM6 on the surface of the tumor cells, and then is endocytosed by the tumor cells into cytoplasmic lysosomes, and the ZAP causes the tumor cells to die. Therefore, using the conjugate formed by the antibody and ZAP to co-incubate with tumor cells, the activity of antibody-mediated endocytosis by tumor cells can be indirectly determined by measuring the viability of the cells.

HPAFII cells were used as target cells, and after centrifugation at 1000 rpm for 4 min at room temperature and resuspension using RPMI1640 basal medium (containing 5% FBS), they were spread in 96-well plates at 2×10³/well with 50 L/well, and cultured for 24 h at 37° C. with 5% CO₂ in a cell culture incubator. Antibodies were diluted using RPMI1640 basal medium (containing 5% FBS) at an initial concentration of 4 nM, and then 10-fold gradient dilutions for a total of 7 concentration gradients, and 25 μL/well were spread. And then Fab-ZAP mouse was diluted to 4 nM using RPMI1640 basal medium (containing 5% FBS), added to the corresponding wells with 25 μL/well, and continued to be cultured in the cell culture incubator for 72 h. Cell viability was calculated using the CCK8 method. CCK-8 staining solution was added with 10 μL/well, and the reaction was protected from light for 2 h, and the OD450 was detected by enzyme-labeled instrument. The % kill was calculated, and the % kill=(NC wells-sample wells)*100/(ZAP wells-blank wells).

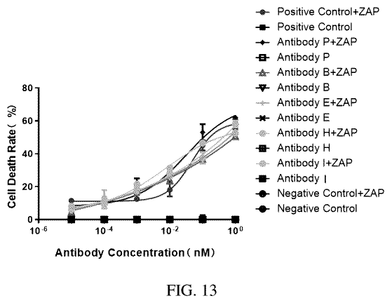

The results are shown in FIG. 13, ZAP alone with antibody group does not affect the viability of HPAFII cells, whereas the conjugate formed by the candidate antibody and ZAP mediates the death of tumor cells in a concentration-dependent manner, and the irrelevant antibody (non-target protein antibody) group does not have such an effect, regardless of the conjugate or toxin control or antibody control alone. This experiment demonstrates that the candidate antibody has a good endocytosis activity and can be used for anti-tumor therapy of coupled small molecule drugs forming ADC drugs.

### Example 15 Affinity Verification of the Antibodies

Equipment: Biacore (GE).

Sensor chip: CM5 chip (GE).

### (1) Immobilisation:

Activator preparation: 400 mM EDC and 100 mM NHS (GE) were mixed immediately before use.

The CM5 sensor chip was activated at a flow rate of 10 μL/min for 420 seconds. The channel was then injected with 30 μg/mL of anti-rat Fc IgG in 10 mM NaAc (pH 4.5) at a flow rate of 10 μL/min. The chip was inactivated with 1 M ethanolamine-hydrochloric acid (GE) for 420 seconds at a flow rate of 10 μL/min.

### (2) Sample Capture:

Candidate antibodies in running buffer 1×HBS-EP+ (10 mM HEPES, 150 mM NaCl, 3 mM EDTA, 0.05% Tween 20, pH 7.4) were captured onto Fc2 with anti-rat Fc IgG at a flow rate of 10 μL/min. The 10 nM human CEACAM5-his protein or CEACAM-6-his or CEACAM6 (AB)-his protein and running buffer were sequentially injected into Fc1-Fc2 at a flow rate of 30 min, bound for 180 seconds, and then dissociated for 3600 seconds. 10 mM glycine (pH 1.5) was injected as regeneration buffer after each dissociation.

### (3) Regeneration:

The chip was regenerated with 10 mM glycine (pH 1.5).

### (4) Data Analysis:

Result plots for the reference channel Fc1 and the buffer channel were subtracted from the test result plots. The experimental data fit the 1:1 binding model. The molecular weight of 110 KDa was used to calculate the molar concentration of CEACAM5 protein, 55 KDa for human CEACAM6-his, and 55 KDa for human CEACAM6 (AB)-his.

TABLE 4
Affinity KD (M) of the antibody proteins

| | To human | To human | To human |
|---|---|---|---|
| Antibody | CEACAM5 | CEACAM6 | CEACAM6(AB) |
| Positive Control | 5.05E−09 | 6.80E−09 | 1.74E−08 |
| Antibody 13 | 1.36E−09 | 3.74E−08 | 4.51E−09 |
| Antibody 15 | 7.64E−09 | 3.83E−09 | <1.0E−12 |
| Antibody B | <1.0E−12 | 1.97E−08 | 1.17E−07 |

### Example 16 Immunohistochemical IHC

Studies have shown that certain tumor tissues, especially colon cancer, highly express CEACAM5 and CEACAM6, while healthy tissues at the corresponding sites also have a certain level of expression. Studies have shown that tumors express proteins that are different from those in normal tissues due to metabolic abnormalities. NEO-201, as an antibody that binds CEACAM5 and CEACAM6, weakly binds to healthy tissues and has a higher affinity for CEACAM5 and CEACAM6 in tumor tissues. Taking advantage of the differential binding of target proteins in tumor tissues and normal tissues, the candidate antibodies were used as primary antibodies, and IHC method was used to identify the antibodies with differential binding. Paraffin-embedded tissue sections are taken from normal and tumor tissues to form a tissue matrix, known as a tissue microarray. Catalog reagent antibodies that bind both CEACAM5 and CEACAM6 are used as target protein expression and localization analyses in tumor and normal tissues.

The results are shown in Table 5, which shows that the negative control (IgG isotype control) does not have any tissue expression, while CEACAM5 and CEACAM6 have a wide range of expression in tumor and normal tissues, with tumor tissues having higher expression levels. The NEO-201 antibody has been shown to bind to CEACAM5 and CEACAM6 proteins in vitro, as well as to tumor cell lines with high expression of CEACAM5 and CEACAM6, whereas in this assay differential binding to tumor tissues versus normal tissues is observed. Compared with the catalog reagent antibodies, NEO-201 has weak binding to normal tissues and almost no binding in healthy stomach, pancreas, and liver tissues, while it has similar or even stronger binding in including stomach and colon cancers. The candidate antibodies also have similar differential binding.

TABLE 5
Differential binding of antibodies by IHC assays

| | Stomach/ | | Pancreas/ | | Esophagus/ |
|---|---|---|---|---|---|
| | Stomach | Colon/Colon | Pancreatic | Liver/liver | Esophageal |
| Sample | Cancer | Cancer | Cancer | cancer | Cancer |
| Negative Control | −/− | −/− | −/− | −/− | −/− |
| Catalog Reagent | ++/++ | +++/++++ | ++/++ | +/+ | ++/++ |
| Antibody | | | | | |
| BMK(NEO-201) | −/++ | +/+++++ | −/++ | −/− | +/+ |
| Antibody 2 | −/++ | +/+++++ | −/++ | −/− | +/+ |
| Antibody 15 | −/+ | +/+++++ | −/++ | −/− | +/+ |
| Antibody 13 | −/+ | +/++++ | −/+ | −/− | +/+ |

Note:
“−” represents no binding, “+” represents binding, the number of “+” represents the strength of the binding, and “+++++“ represents the strongest binding.

The protection scope of the present invention is not limited to the above embodiments. Without departing from the spirit and scope of the inventive conception, variations and advantages that can be thought of by those skilled in the art are included in the present invention and are protected by the appended claims.

1. An antibody against CEACAM5 and CEACAM6 or antigen-binding fragment thereof, comprising: a heavy chain variable region and a light chain variable region, wherein the heavy chain variable region comprises the heavy chain complementary determining regions HCDR1, HCDR2 and HCDR3, and the light chain variable region comprises the light chain complementary determining regions LCDR1, LCDR2 and LCDR3, wherein,
(a) HCDR1 of the heavy chain variable region is selected from any amino acid sequence of any of SEQ ID NO: 1-14, or a sequence having at least 80%, 85%, 90%, 91%, 92%, 93%, 94%, 95%, 96%, 97%, 98%, 99% or more identity to the amino acid sequence of any of SEQ ID NO: 1-14, or an amino acid sequence having one or more conserved amino acid mutations as compared to the amino acid sequence of any of SEQ ID NO: 1-14;
(b) HCDR2 of the heavy chain variable region is selected from any amino acid sequence of any of SEQ ID NO: 15-28, 121, or a sequence having at least 80%, 85%, 90%, 91%, 92%, 93%, 94%, 95%, 96%, 97%, 98%, 99% or more identity to the amino acid sequence of any of SEQ ID NO: 15-28, 121, or an amino acid sequence having one or more conserved amino acid mutations as compared to the amino acid sequence of any of SEQ ID NO: 15-28, 121;
(c) HCDR3 of the heavy chain variable region is selected from any amino acid sequence of any of SEQ ID NO: 29-41, or a sequence having at least 80%, 85%, 90%, 91%, 92%, 93%, 94%, 95%, 96%, 97%, 98%, 99% or more identity to the amino acid sequence of any of SEQ ID NO: 29-41, or an amino acid sequence having one or more conserved amino acid mutations as compared to the amino acid sequence of any of SEQ ID NO: 29-41;
(d) LCDR1 of the light chain variable region is selected from any amino acid sequence of SEQ ID NO: 42-54, or a sequence having at least 80%, 85%, 90%, 91%, 92%, 93%, 94%, 95%, 96%, 97%, 98%, 99% or more identity to any amino acid sequence of SEQ ID NO: 42-54, or an amino acid sequence having one or more conserved amino acid mutations in comparison to any amino acid sequence of SEQ ID NO: 42-54;
(e) LCDR2 of the light chain variable region is selected from any amino acid sequence of SEQ ID NO: 55-65, or a sequence having at least 80%, 85%, 90%, 91%, 92%, 93%, 94%, 95%, 96%, 97%, 98%, 99% or more identity to any amino acid sequence of SEQ ID NO: 55-65, or an amino acid sequence having one or more conserved amino acid mutations in comparison to any amino acid sequence of SEQ ID NO: 55-65; and/or
(f) LCDR3 of the light chain variable region is selected from any amino acid sequence of SEQ ID NO: 66-79, or a sequence having at least 80%, 85%, 90%, 91%, 92%, 93%, 94%, 95%, 96%, 97%, 98%, 99% or more identity to any amino acid sequence of SEQ ID NO: 66-79, or an amino acid sequence having one or more conserved amino acid mutations in comparison to any amino acid sequence of SEQ ID NO: 66-79.

2. The antibody against CEACAM5 and CEACAM6 or antigen-binding fragment thereof of claim 1, wherein HCDR1, HCDR2, HCDR3 of the heavy chain variable region, and LCDR1, LCDR2, LCDR3 of the light chain variable region are selected from the amino acid sequences of any of the following (1)-(17):
(1) HCDR1 as shown in SEQ ID NO: 1, HCDR2 as shown in SEQ ID NO: 15, HCDR3 as shown in SEQ ID NO: 29, LCDR1 as shown in SEQ ID NO: 42, LCDR2 as shown in SEQ ID NO: 55, and LCDR3 as shown in SEQ ID NO: 66;
(2) HCDR1 as shown in SEQ ID NO: 2, HCDR2 as shown in SEQ ID NO: 16, HCDR3 as shown in SEQ ID NO: 30, LCDR1 as shown in SEQ ID NO: 43, LCDR2 as shown in SEQ ID NO: 56, and LCDR3 as shown in SEQ ID NO: 67;
(3) HCDR1 as shown in SEQ ID NO: 3, HCDR2 as shown in SEQ ID NO: 17, HCDR3 as shown in SEQ ID NO: 31, LCDR1 as shown in SEQ ID NO: 44, LCDR2 as shown in SEQ ID NO: 57, and LCDR3 as shown in SEQ ID NO: 68;
(4) HCDR1 as shown in SEQ ID NO: 4, HCDR2 as shown in SEQ ID NO: 18, HCDR3 as shown in SEQ ID NO: 32, LCDR1 as shown in SEQ ID NO: 45, LCDR2 as shown in SEQ ID NO: 58, and LCDR3 as shown in SEQ ID NO: 69;
(5) HCDR1 as shown in SEQ ID NO: 5, HCDR2 as shown in SEQ ID NO: 19, HCDR3 as shown in SEQ ID NO: 33, LCDR1 as shown in SEQ ID NO: 46, LCDR2 as shown in SEQ ID NO: 59, and LCDR3 as shown in SEQ ID NO: 70;
(6) HCDR1 as shown in SEQ ID NO: 6, HCDR2 as shown in SEQ ID NO: 20, HCDR3 as shown in SEQ ID NO: 34, LCDR1 as shown in SEQ ID NO: 47, LCDR2 as shown in SEQ ID NO: 60, and LCDR3 as shown in SEQ ID NO: 67;
(7) HCDR1 as shown in SEQ ID NO: 6, HCDR2 as shown in SEQ ID NO: 20, HCDR3 as shown in SEQ ID NO: 34, LCDR1 as shown in SEQ ID NO: 48, LCDR2 as shown in SEQ ID NO: 61, and LCDR3 as shown in SEQ ID NO: 71;
(8) HCDR1 as shown in SEQ ID NO: 7, HCDR2 as shown in SEQ ID NO: 21, HCDR3 as shown in SEQ ID NO: 35, LCDR1 as shown in SEQ ID NO: 49, LCDR2 as shown in SEQ ID NO: 62, and LCDR3 as shown in SEQ ID NO: 72;
(9) HCDR1 as shown in SEQ ID NO: 8, HCDR2 as shown in SEQ ID NO: 22, HCDR3 as shown in SEQ ID NO: 36, LCDR1 as shown in SEQ ID NO: 50, LCDR2 as shown in SEQ ID NO: 63, and LCDR3 as shown in SEQ ID NO: 73;
(10) HCDR1 as shown in SEQ ID NO: 9, HCDR2 as shown in SEQ ID NO: 23, HCDR3 as shown in SEQ ID NO: 30, LCDR1 as shown in SEQ ID NO: 51, LCDR2 as shown in SEQ ID NO: 56, and LCDR3 as shown in SEQ ID NO: 74;
(11) HCDR1 as shown in SEQ ID NO: 10, HCDR2 as shown in SEQ ID NO: 24, HCDR3 as shown in SEQ ID NO: 37, LCDR1 as shown in SEQ ID NO: 50, LCDR2 as shown in SEQ ID NO: 63, and LCDR3 as shown in SEQ ID NO: 75;
(12) HCDR1 as shown in SEQ ID NO: 11, HCDR2 as shown in SEQ ID NO: 24, HCDR3 as shown in SEQ ID NO: 37, LCDR1 as shown in SEQ ID NO: 50, LCDR2 as shown in SEQ ID NO: 63, and LCDR3 as shown in SEQ ID NO: 76;
(13) HCDR1 as shown in SEQ ID NO: 12, HCDR2 as shown in SEQ ID NO: 25, HCDR3 as shown in SEQ ID NO: 38, LCDR1 as shown in SEQ ID NO: 52, LCDR2 as shown in SEQ ID NO: 64, and LCDR3 as shown in SEQ ID NO: 67;
(14) HCDR1 as shown in SEQ ID NO: 2, HCDR2 as shown in SEQ ID NO: 26, HCDR3 as shown in SEQ ID NO: 39, LCDR1 as shown in SEQ ID NO: 51, LCDR2 as shown in SEQ ID NO: 56, and LCDR3 as shown in SEQ ID NO: 77;
(15) HCDR1 as shown in SEQ ID NO: 13, HCDR2 as shown in SEQ ID NO: 27, HCDR3 as shown in SEQ ID NO: 40, LCDR1 as shown in SEQ ID NO: 53, LCDR2 as shown in SEQ ID NO: 57, and LCDR3 as shown in SEQ ID NO: 78;
(16) HCDR1 as shown in SEQ ID NO: 14, HCDR2 as shown in SEQ ID NO: 28, HCDR3 as shown in SEQ ID NO: 41, LCDR1 as shown in SEQ ID NO: 54, LCDR2 as shown in SEQ ID NO: 65, and LCDR3 as shown in SEQ ID NO: 79; and
(17) HCDR1 as shown in SEQ ID NO: 2, HCDR2 as shown in SEQ ID NO: 121, HCDR3 as shown in SEQ ID NO: 30, LCDR1 as shown in SEQ ID NO: 43, LCDR2 as shown in SEQ ID NO: 56, and LCDR3 as shown in SEQ ID NO: 67.

3. An antibody against CEACAM5 and CEACAM6 or antigen-binding fragment thereof, comprising: a heavy chain variable region and a light chain variable region, wherein,
(a) the heavy chain variable region has an amino acid sequence of any of the amino acids that are given in SEQ ID NO: 80-94, 122-125, or a sequence having at least 80%, 85%, 90%, 91%, 92%, 93%, 94%, 95%, 96%, 97%, 98%, 99% or more identity to an amino acid sequence that are given in SEQ ID NO: 80-94, 122-125 or an amino acid sequence having one or more conserved amino acid mutations in comparison to any of the amino acid sequences of SEQ ID NO: 80-94, 122-125;
(b) the light chain variable region has an amino acid sequence of any of the amino acids that are given in SEQ ID NO: 95-110, 126-128, or a sequence having at least 80%, 85%, 90%, 91%, 92%, 93%, 94%, 95%, 96%, 97%, 98%, 99% or more identity to an amino acid sequence that are given in SEQ ID NO: 95-110, 126-128, or an amino acid sequence having one or more conserved amino acid mutations in comparison to any of the amino acid sequences of SEQ ID NO: 95-110, 126-128.

4. The antibody against CEACAM5 and CEACAM6 or antigen-binding fragment thereof of claim 3, wherein the heavy chain variable region and the light chain variable region are selected from any of the following amino acid sequences (1)-(28):
(1) SEQ ID NO: 80 and SEQ ID NO: 95;
(2) SEQ ID NO: 81 and SEQ ID NO: 96;
(3) SEQ ID NO: 82 and SEQ ID NO: 97;
(4) SEQ ID NO: 83 and SEQ ID NO: 98;
(5) SEQ ID NO: 84 and SEQ ID NO: 99;
(6) SEQ ID NO: 85 and SEQ ID NO: 100;
(7) SEQ ID NO: 85 and SEQ ID NO: 101;
(8) SEQ ID NO: 86 and SEQ ID NO: 102;
(9) SEQ ID NO: 87 and SEQ ID NO: 103;
(10) SEQ ID NO: 88 and SEQ ID NO: 104;
(11) SEQ ID NO: 89 and SEQ ID NO: 105;
(12) SEQ ID NO: 90 and SEQ ID NO: 106;
(13) SEQ ID NO: 91 and SEQ ID NO: 107;
(14) SEQ ID NO: 92 and SEQ ID NO: 108;
(15) SEQ ID NO: 93 and SEQ ID NO: 109;
(16) SEQ ID NO: 94 and SEQ ID NO: 110;
(17) SEQ ID NO: 122 and SEQ ID NO: 126;
(18) SEQ ID NO: 122 and SEQ ID NO: 127;
(19) SEQ ID NO: 122 and SEQ ID NO: 128;
(20) SEQ ID NO: 123 and SEQ ID NO: 126;
(21) SEQ ID NO: 123 and SEQ ID NO: 127;
(22) SEQ ID NO: 123 and SEQ ID NO: 128;
(23) SEQ ID NO: 124 and SEQ ID NO: 126;
(24) SEQ ID NO: 124 and SEQ ID NO: 127;
(25) SEQ ID NO: 124 and SEQ ID NO: 128;
(26) SEQ ID NO: 125 and SEQ ID NO: 126;
(27) SEQ ID NO: 125 and SEQ ID NO: 127; and
(28) SEQ ID NO: 125 and SEQ ID NO: 128.

5. The antibody against CEACAM5 and CEACAM6 or antigen-binding fragment thereof of any one of claim 3, wherein the antibody is a mouse-derived antibody, a chimeric antibody, a humanized antibody or a fully human antibody.

6. The antibody against CEACAM5 and CEACAM6 or antigen-binding fragment thereof of claim 5, wherein the antibody is a monoclonal antibody.

7. The antibody against CEACAM5 and CEACAM6 or antigen-binding fragment thereof of claim 6, further comprising an Fc region, and the Fc region is selected from the group consisting of: mouse IgG1, IgG2a, IgG2b and/or IgG3, or rat IgG1, IgG2a, IgG2b and/or IgG2c.

8. The antibody against CEACAM5 and CEACAM6 or antigen-binding fragment thereof of claim 6, further comprising an Fc region, and the Fc region is selected from the group consisting of: human IgG1, IgG2, IgG3 and/or IgG4 or an amino acid sequence in the Fc region with one or more amino acid mutations with human IgG1, IgG2, IgG3, IgG4.

9. (canceled)

10. A multifunctional fusion protein, comprising the antibody against CEACAM5 and CEACAM6 or antigen-binding fragment thereof of claim 1.

11. The multifunctional fusion protein of claim 10, further comprising one or more third antibodies or antigen-binding portions thereof that bind specifically to other antigens.

12. The multifunctional fusion protein of claim 11, wherein the antigen binding the third antibody or antigen-binding portion thereof is selected from the group consisting of: a tumor-associated antigen (TAA) or an immune checkpoint.

13. The multifunctional fusion protein of claim 12, wherein the immune checkpoint is PD-L1, CTLA4, PD-L2, PD-1, 4-1BB, CD47, TIGIT, GITR, TIM3, ILT4, TNFR2, TREM2, LAG3, CD27, B7H3 or B7H4.

14. The multifunctional fusion protein of claim 10, further comprising a cytokine.

15. The multifunctional fusion protein of claim 14, wherein the cytokine is selected from the group consisting of: IL-1, IL-2, IL-2 Rα, IL-2 Rβ, IL-3, IL-3 Rα, IL-4, IL-4 Rα, IL-5, IL-5 Rα, IL-6, IL-6 Rα, IL-7, IL-7 Rα, IL-8, IL-9, IL-9 Rα, IL-10, IL-10R1, IL-10R2, IL-11, IL-11 Rα, IL-12, IL-12 Rα, IL-12 Rβ2, IL-12 Rβ1, IL-13, IL-13 Rα, IL-13 Rα2, IL-14, IL-15, IL-15Rα sushi, IL-16, IL-17, IL-18, IL-19, IL-20, IL-20R1, IL-20R2, IL-21, IL-21 Rα, IL-22, IL-23, IL-23R, IL-27 R, IL-31 R, TGF, VEGF, IFNγ, IFNα or GM-CSF.

16. (canceled)

17. (canceled)

18. (canceled)

19. (canceled)

20. (canceled)

21. (canceled)

22. (canceled)

23. (canceled)

24. (canceled)
